SEQUENCE LISTING (10 sequences)

SEQ ID NO: 1 - protein, 118 aa, synthetic construct
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARHEWGFGMDVWGQGTTVTVSS

SEQ ID NO: 2 - protein, 119 aa, synthetic construct
DVVMTQSPLSLSVTLGQPASISCRSSLSLVYGDENTYLNWFQQRPGQSPRRLLYKVSDRDSGVPDRFSGSGSGTDFTLKISRVEADDVGVYYCMQGTHWPYTFGQGTKLEIKRTVAAPS

SEQ ID NO: 3 - protein, 8 aa, synthetic construct
GFTFSSYA

SEQ ID NO: 4 - protein, 8 aa, synthetic construct
ISYDGSNK

SEQ ID NO: 5 - protein, 11 aa, synthetic construct
ARHEWGFGMDV

SEQ ID NO: 6 - protein, 10 aa, synthetic construct
SLVYGDENTY

SEQ ID NO: 7 - skipped

SEQ ID NO: 8 - protein, 7 aa, synthetic construct
MQGTHWP

SEQ ID NO: 9 - protein, 219 aa, synthetic construct
DVVMTQSPLSLSVTLGQPASISCRSSLSLVYGDENTYLNWFQQRPGQSPRRLLYKVSDRDSGVPDRFSGSGSGTDFTLKISRVEADDVGVYYCMQGTHWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 219 aa, synthetic construct; identical to SEQ ID NO: 9

(19) United States
(12) Patent Application Publication
(10) Pub. No.: US 2026/0070996 A1
(43) Pub. Date: Mar. 12, 2026

(54) FORMULATIONS FOR ANTI-INSULIN RECEPTOR ANTIBODY AND USES THEREOF

(71) Applicants: REZOLUTE, INC., Redwood City, CA (US); XOMA (US) LLC, Emeryville, CA (US)

(72) Inventors: Alona Teran, Redwood City, CA (US); Mingxiang Li, Emeryville, CA (US); Joel Freeberg, Emeryville, CA (US)

(21) Appl. No.: 18/867,485

(22) PCT Filed: May 19, 2023
(86) PCT No.: PCT/US2023/067250

§ 371 Date: Nov. 20, 2024

Related U.S. Application Data
(60) Provisional application No. 63/344,286, filed on May 20, 2022.

(51) Int. Cl. C07K 16/28; A61K 39/395; A61K 47/18; A61K 47/20; A61K 47/26

(52) U.S. Cl. CPC C07K 16/2869; A61K 39/39591; A61K 47/183; A61K 47/20; A61K 47/26; C07K 2317/92

(57) ABSTRACT

The present disclosure relates, in general, to formulations comprising anti-insulin receptor antibody RZ 358 and use thereof to treat hyperinsulinemia disorders.

### CROSS-REFERENCE TO RELATED APPLICATIONS

The present application claims the priority benefit of U.S. Provisional Patent Application 63/344,286, filed May 20, 2022, incorporated herein in its entirety.

### FIELD OF THE DISCLOSURE

The present disclosure relates, in general, to formulations of an antibody specific for the insulin receptor in the treatment and prevention of hypoglycemia and conditions related to hyperinsulinemia, such as congenital hyperinsulinemia.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “57847_Seqlisting.hml”, which was created on May 16, 2023, and is 9,676 bytes in size. The subject matter of the Sequence Listing is incorporated herein in its entirety by reference.

### BACKGROUND

Insulin is the major hormone for lowering blood glucose levels. The first step in insulin action is the binding of the hormone to the insulin receptor (INSR), an integral membrane glycoprotein, also designated as CD220 or HHF5. When insulin binds to the INSR, the receptor is activated by tyrosine autophosphorylation and the INSR tyrosine kinase phosphorylates various effector molecules, including the insulin receptor substrate-1 (IRS-1), leading to hormone action (Ullrich et al, Nature 313: 756-761, 1985; Goldfine et al, Endocrine Reviews 8: 235-255, 1987; White and Kahn, Journal Biol. Chem. 269: 1-4, 1994). IRS-1 binding, and phosphorylation eventually leads to an increase in the high affinity glucose transporter (Glut4) molecules on the outer membrane of insulin-responsive tissues, including muscle cells and adipose tissue, and to an increase in the uptake of glucose from blood into these tissues. Glut4 mediates the transport of glucose into the cell and a decrease in blood glucose levels.

Abnormal increases in insulin secretion can lead to hypoglycemia or low blood sugar, a state that may result in significant morbidities including epilepsy and cerebral damage. Drug-induced hypoglycemia can result from administration of sulfonylurea drugs or from an overdose of insulin. A number of rare medical conditions feature non-drug-induced, endogenous hyperinsulinemic hypoglycemia, i.e., low blood glucose caused by the body's excessive production of insulin. These conditions include congenital hyperinsulinism, insulinoma, and hyperinsulinemic hypoglycemia following gastric bypass surgery.

RZ358 is a monoclonal antibody specific for insulin receptor (INSR) that modulates binding of insulin to the insulin receptor and binds allosterically to INSR without blocking the binding of insulin to the insulin receptor. In a Phase 1 trial, RZ358 was shown to reduce glucose levels in healthy volunteers (Johnson et al., J Clin Endocrinol Metab. 2017 102(8):3021-3028), and in a later study shown to reduced daily periods of hypoglycemia and correct nighttime hypoglycemia in Post Gastric Bypass Hypoglycemia (PGBH) Patients (Hu et al., Journal of the Endocrine Society, Volume 5, Issue Supplement 1, April-May 2021, Pages A328-A329). A recent Phase 2b trial demonstrated improvements in hypoglycemia in congenital hyperinsulinism (CHI) patients receiving antibody therapy (Thornton et al., RZ358 in Congenital Hyperinsulinism: Results from a Multi-Center, Global, Phase 2b Study (RIZE), Pediatric Endocrine Society Annual Meeting 2022).

### SUMMARY

Contemplated herein are formulations for anti-INSR antibodies, e.g., RZ 358, which provide stability of the antibody over time at different storage conditions.

The disclosure provides a composition comprising an antibody that specifically binds insulin receptor (INSR), at least one amino acid or a salt thereof, a surfactant, and a sugar alcohol, wherein the anti-INSR antibody comprises (A) a light chain variable domain comprising: (i) a light chain CDR1 sequence comprising the amino acid sequence set forth in SEQ ID NO: 6; (ii) a light chain CDR2 sequence comprising the amino acid sequence set forth in SEQ ID NO: 7; and (iii) a light chain CDR3 sequence comprising the amino acid sequence set forth in SEQ ID NO: 8; and (B) a heavy chain variable domain comprising: (i) a heavy chain CDR1 sequence comprising the amino acid sequence set forth in SEQ ID NO: 3; (ii) a heavy chain CDR2 sequence comprising the amino acid sequence set forth in SEQ ID NO: 4, and (iii) a heavy chain CDR3 sequence comprising the amino acid sequence set forth in SEQ ID NO: 5.

In various embodiments, the anti-INSR antibody comprises: (A) a light chain variable domain comprising a sequence of amino acids at least 80% identical to SEQ ID NO: 2; or (B) a heavy chain variable domain comprising a sequence of amino acids that is at least 80% identical to SEQ ID NO: 1; or (C) a light chain variable domain of (A) and a heavy chain variable domain of (B). In various embodiments, the heavy chain variable region amino acid sequence is at least 85%, 90%, or 95% or more identical to SEQ ID NO: 1. In various embodiments, the light chain variable region amino acid sequences is at least 85%, 90%, or 95% or more identical to SEQ ID NO: 2.

In various embodiments, the anti-INSR antibody comprises a heavy chain variable region comprising the amino acid sequence of SEQ ID NO: 1, and a light chain variable region comprising the amino acid sequence of SEQ ID NO: 2.

In various embodiments, the anti-INSR antibody comprises a heavy chain comprising the amino acid sequence of SEQ ID NO: 9, and a light chain comprising the amino acid sequence of SEQ ID NO: 10.

In various embodiments, the anti-INSR antibody is an IgG2 antibody.

In various embodiments, the anti-INSR antibody is RZ 358. In various embodiments, RZ358 comprises (A) a light chain variable domain comprising: (i) a light chain CDR1 sequence comprising the amino acid sequence set forth in SEQ ID NO: 6; (ii) a light chain CDR2 sequence comprising the amino acid sequence set forth in SEQ ID NO: 7; and (iii) a light chain CDR3 sequence comprising the amino acid sequence set forth in SEQ ID NO: 8; and (B) a heavy chain variable domain comprising: (i) a heavy chain CDR1 sequence comprising the amino acid sequence set forth in SEQ ID NO: 3; (ii) a heavy chain CDR2 sequence comprising the amino acid sequence set forth in SEQ ID NO: 4, and (iii) a heavy chain CDR3 sequence comprising the amino acid sequence set forth in SEQ ID NO: 5; or a heavy chain variable region comprising the amino acid sequence of SEQ ID NO: 1, and a light chain variable region comprising the amino acid sequence of SEQ ID NO: 2; or a heavy chain comprising the amino acid sequence of SEQ ID NO: 9, and a light chain comprising the amino acid sequence of SEQ ID NO: 10.

In various embodiments, the amino acid or salt thereof is selected from the group consisting of histidine, histidine HCL, methionine, and arginine. In various embodiments, the amino acid is histidine. In various embodiments, the histidine is at a concentration of from about 4 mM to about 25 mM or about 10 mM to about 20 mM. In various embodiments, the histidine is at a concentration of about 4±1 mM.

In various embodiments, the amino acid is histidine HCL. In various embodiments, the histidine HCL is at a concentration of from about 4 mM to about 25 mM or about 10 mM to about 20 mM. In various embodiments, the histidine HCL is at a concentration of about 6±1 mM.

In various embodiments, the amino acid is methionine. In various embodiments, the methionine is at a concentration of from about 4 mM to about 25 mM or about 10 mM to about 20 mM. In various embodiments, the methionine is at a concentration of about 10±2 mM.

In various embodiments, the surfactant is a polysorbate. In various embodiments, the polysorbate is polysorbate 20 or polysorbate 80 or a mixture thereof. In various embodiments, the surfactant is at a concentration of about 0.002% (w/v) to about 0.02% (w/v). In various embodiments, the composition comprises about 0.005% (w/v), 0.010% (w/v), 0.015% (w/v), or 0.02% (w/v) surfactant. In various embodiments, the composition comprises about 0.01% (w/v)±0.0025% (w/v) surfactant, optionally wherein the surfactant is polysorbate 20 or polysorbate 80 or a mixture thereof.

In various embodiments, the sugar alcohol is selected from the group consisting of sucrose, sorbitol and mannitol.

In various embodiments, the sugar alcohol is sorbitol. In various embodiments, the sorbitol is at a concentration of from about 100 mM to about 350 mM or about 200 mM to about 300 mM. In various embodiments, the sorbitol is at a concentration of about 270±30 mM.

In various embodiments, the sugar alcohol is mannitol. In various embodiments, the mannitol is at a concentration of from about 100 mM to about 350 mM or about 200 mM to about 300 mM. In various embodiments, the mannitol is at a concentration of about 270±30 mM.

In various embodiments, the anti-INSR antibody is present in the composition at a concentration from about 20 mg/ml to about 200 mg/mL. In various embodiments, anti-INSR antibody is present in the composition at a concentration from about 50 mg/ml to about 150 mg/mL. In various embodiments, anti-INSR antibody is present in the composition at a concentration of about 80 mg/mL to about 120 mg/mL.

In various embodiments, the composition is a liquid. In various embodiments, the composition is lyophilized. In various embodiments, the composition is a liquid reconstituted from a lyophilized form.

In various embodiments, the pH is less than about 6.5. In various embodiments, the pH is about 5.0 to about 6.5. In various embodiments, the pH is about 5.5 to about 5.9. In various embodiments, the pH is about 5.8.

In various embodiments, the composition is characterized by a viscosity of about 2 cP to about 10 cP, at 25° C., wherein the concentration of the anti-INSR antibody is about 100 mg/ml or less.

In various embodiments, the composition is isotonic or has an osmolality in a range of about 200 mOsm/kg to about 500 mOsm/kg, or about 225 mOsm/kg to about 400 mOsm/kg, or about 250 mOsm/kg to about 400 mOsm/kg.

In various embodiments, the composition comprises less than about 10% impurities after about 24 months to about 36 months of storage at 2° C. to 8° C. as determined by reduced sodium dodecyl sulfate capillary electrophoresis (rCE-SDS) analysis. Impurities include degradation products, high molecular weight species, low molecular weight species, oxidation species, or aggregates.

In various embodiments, less than 10% of the antibody is degraded after about 24 months to about 36 months of storage at 2° C. to 8° C. as determined by reduced sodium dodecyl sulfate capillary electrophoresis (rCE-SDS) analysis. In various embodiments, the composition comprises less than about 5% degradation products after about 24 months to about 36 months of storage at 2° C. to 8° C. as determined by reduced sodium dodecyl sulfate capillary electrophoresis (rCE-SDS) analysis.

In various embodiments, less than 15% of the antibody is oxidized or aggregated after about 3 months of storage at 40° C. as determined by Hydrophobic Interaction Chromatography (HIC) or size exclusion chromatography (SEC).

In various embodiments, less than 30% of the antibody is detected in the acidic peak after about 24 months to about 36 months of storage at 2° C. to 8° C. as determined by cIEX-UHPLC analysis. In various embodiments, approximately 8-20% of the antibody is detected in the acidic peak after about 24 months to about 36 months of storage at 2° C. to 8° C. as determined by cIEX-UHPLC analysis. In various embodiments, less than 22% of the antibody is detected in the basic peak after about 24 months to about 36 months of storage at 2° C. to 8° C. as determined by cIEX-UHPLC analysis. In various embodiments, about 5-20% of the antibody is detected in the basic peak after about 24 months to about 36 months of storage at 2° C. to 8° C. as determined by cIEX-UHPLC analysis.

In various embodiments, the composition comprises less than 5% high molecular weight species after about 24 months to about 36 months of storage at 2° C. to 8° C. as determined by SE-UHPLC. In various embodiments, the composition comprises less than 5% low molecular weight species after about 24 months to about 36 months of storage at 2° C. to 8° C. as determined by SE-UHPLC.

In various embodiments, the composition comprises less than 18% antibody in oxidized form after about 24 months to about 36 months of storage at 2° C. to 8° C. as determined by HIC-HPLC.

In various embodiments, the potency of the antibody composition is at least about 75% to about 120% after about 24 months to about 36 months of storage at 2° C. to 8° C. as determined by bioassay. In various embodiments, the bioassay is a phosphorylated AKT (pAKT) assay.

Provided herein is a composition comprising about 20-100 mg/mL of an anti-INSR antibody, 0.01% (w/v) polysorbate 20, about 250 mM to about 300 mM sorbitol, about 5 mM to about 15 mM methionine, and about 5 mM to about 15 mM histidine, wherein the composition has a pH of about 5.8. In various embodiments, the anti-INSR antibody is RZ358.

Further contemplated is an article of manufacture comprising the composition as described herein optionally, comprising about 0.5 mL to about 5 mL, or about 1 mL to about 3 mL of the composition.

Also contemplated is a method for treating a condition associated with hyperinsulinemia or excess insulin signaling in a subject in need thereof comprising administering to the subject a therapeutically effective amount of the composition comprising the anti-INSR antibody as described herein. In various embodiments, condition is selected from the group consisting of: hypoglycemia, insulin sensitivity, cancer, insulinoma, Kaposi's sarcoma, insulin overdose, nesidioblastosis (KATP-HI Diffuse Disease, KATP-HI Focal Disease, or “PHHI”), GDH-HI (Hyperinsulinism/Hyperammonaemia Syndrome (HI/HA), leucine-sensitive hypoglycemia, diazoxide-sensitive hypoglycemia, islet cell dysregulation syndrome, idiopathic hypoglycemia of infancy, Persistent Hyperinsulinemic Hypoglycemia of Infancy (PHHI), Congenital Hyperinsulinism, acute hypoglycemia due to renal failure, chronic hypoglycemia due to renal failure, and hypoglycemia due to chronic kidney disease. In various embodiments, the disease is Congenital Hyperinsulinism.

In various embodiments, the composition is administered daily, every 2 days, every 3 days, weekly, every 2 weeks, every 3 weeks, twice monthly, monthly, every 2 months, every 3 months or every 6 months. In various embodiments, the composition is administered for a period of at least 1 month, 2 months, 3 months, 4 months, 6 months, 9 months, 1 year or more.

In various embodiments, the composition is administered intravenously. In various embodiments, the composition is administered subcutaneously.

The disclosure also provides a composition comprising the anti-INSR antibody as described herein for treating a condition associated with hyperinsulinemia or excess insulin signaling in a subject in need thereof.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawings and/or claims.

### BRIEF DESCRIPTION OF THE DRAWINGS

FIG. 1 depicts DSC thermograms of RZ 358 at different pH in citrate buffered saline formulations.

FIG. 2 shows SEC chromatograms (UV214 nm) of freeze-thaw stressed samples in pH 4.0 (upper panel) and 7.0 (lower panel) citrate buffer saline formulation.

FIG. 3 shows SEC (upper panel) and HIC (lower panel) chromatograms (UV280 nm) for the three month-40° C. stability samples formulated in different pH from pH 5.0 to pH 7.0 in citrate buffered saline.

FIG. 4 shows WCX chromatograms for RZ 358 stability samples in different pH formulations pH 5.0 to pH 7.0 stored at 40° C. for 1 month (upper panel) and 30° C. for 3 months (lower panel).

FIG. 5 illustrates the changes in total acidic (upper graph) and total basic (lower graph) species at 30° C. for RZ 358 stability samples formulated at different pH values from pH 5.0 to pH 7.0.

FIG. 6 shows SEC (upper panel) and HIC-HPLC (lower panel) chromatograms for RZ 358 stability samples at different pH formulations pH 5.0 to pH 7.0 stored at 40° C. for 3 months.

FIG. 7 provides the outcome of agitation studies showing SEC-HPLC chromatograms of RZ 358 samples formulated in 0.002% (upper panel) and 0.01% (lower panel) PS20 (Samples were shaken at 1,000 RPM/25° C. for up to 8 days.)

FIG. 8 shows particle concentration (counts per mL) for 10 μm (upper panel) and 25 μm (lower panel) for samples with different polysorbate 20 concentrations stressed by up to 5 cycles of freeze-thaw (−70° C./RT).

FIG. 9 depicts DSC thermograms for XMET D samples formulated in different formulations at pH 6 (Cit/NaCl=10 mM NaCit. 150 mM NaCl; H/R=10 mM L-histidine, 150 mM L-arginine; mannitol=10 mM L-histidine, 10 mM L-methionine, 270 mM mannitol; sorbitol=10 mM Lhistidine, 10 mM L-methionine, 270 mM sorbitol).

FIG. 10 shows formation of “pre-main HIC peaks” (oxidation) at 40° C. for RZ 358 stability samples formulated in three different formulations (Arg.=10 mM L-histidine, 150 mM L-arginine; Man.=10 mM L-histidine, 10 mM L-methionine, 270 mM mannitol; Sorb.=10 mM L-histidine, 10 mM L-methionine, 270 mM sorbitol).

FIG. 11 shows chromatograms for stability samples stored at 40° C. for 3 month (upper panel=SEC; lower panel=HIC; (1) blue=10 mM His, 150 mM L-arg.; (2) red=10 mM His, 10 mM Met, 270 mM mannitol; (3) green=10 mM His, 10 mM Met, 270 mM sorbitol).

FIG. 12 shows chromatograms of stability samples stored at 40° C. for 2 months in different formulations with various concentrations of sorbitol, 90-270 mM (upper panel=SEC; lower panel=HIC).

FIG. 13 shows chromatograms for stability samples stored at 40° C. for 3 months in formulations with different concentrations of methionine, 0-10 mM (upper panel=HIC; lower panel=SEC).

FIG. 14A-14B shows the long term stability at 2° C. to 8° C. of 80 mg/mL RZ358 in 10 mM L-Histidine, 10 mM L-Methionine, 270 mM Sorbitol, 0.01% (w/w) Polysorbate 20 between pH 5.4 and pH 6.3 (FIG. 14A) and effects on protein concentration at pH 5.8 (FIG. 14B) for up to 60 months.

FIG. 15A-15B show total impurities of 80 mg/mL RZ358 in 10 mM L-Histidine, 10 mM L-Methionine, 270 mM Sorbitol, 0.01% (w/w) Polysorbate 20 between pH 5.8 over time at reduced (FIG. 15A) and non-reduced (FIG. 15B) conditions.

FIG. 16A shows reduced CE-SDS analysis of the main peak fraction. FIGS. 16B-16C show cation-exchange chromatography analysis of antibody detected in the main peaks M1 and M2 and the M1/M2 ratio is shown in FIG. 16D.

FIG. 17A shows the CEX-UHPLC acidic peak fraction of the 80 mg/mL RZ358 in 10 mM L-Histidine, 10 mM L-Methionine, 270 mM Sorbitol, 0.01% (w/w) Polysorbate 20 and FIG. 17B shows the CEX-UHPLC basic peak fraction.

FIG. 18A-18C shows the degradation of antibody during storage at 2-8° C. for 60 months, as illustrated by SE-UHPLC analysis of monomeric species (FIG. 18A), % high molecule weight species (FIG. 18B) or % low molecular weight species (FIG. 18C). FIG. 18D shows % oxidized species under the same storage conditions.

FIG. 19 shows the potency of the antibody formulation after storage at 2-8° C. for 60 months as assessed by cell bioassay.

FIG. 20 shows the number of particles per container ≥10 μm, RZ358 DP at 2° to 8° C. formed over 60 months.

### DETAILED DESCRIPTION

The present disclosure provides formulations of a monoclonal antibody specific for the insulin receptor, RZ358, that are stable over long term storage with minimal impurities formed and relative potency of the antibody maintained during storage. Such stable antibody formulations are useful in the treatment of disorders associated with insulin resistance and conditions related to hyperinsulinemia.

### Definitions

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The compositions and methods are contemplated to include embodiments including any combination of one or more of the additional optional elements, features, and steps further described below (including those shown in the figures), unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It should be understood that every maximum numerical limitation given throughout this specification includes as alternative aspects ranges formed with every corresponding lower numerical limitation, as if such ranges were expressly written. Every minimum numerical limitation given throughout this specification will include as alternative aspects ranges formed with every higher numerical limitation, as if such ranges were expressly written. Every numerical range given throughout this specification will include every narrower numerical range that falls within such broader numerical range, as if such narrower numerical ranges were all expressly written herein. The dimensions and values disclosed herein should be understood to include disclosure of both the recited value and the corresponding exact numerical, e.g., a value described as “about 10 mM” should be understood to include, as an alternative disclosure, “10 mM.”

All patents, publications and references cited herein are hereby fully incorporated by reference. In case of conflict between the present disclosure and incorporated patents, publications and references, the present disclosure should control.

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “antibody” is used in the broadest sense and includes fully assembled antibodies, tetrameric antibodies, monoclonal antibodies, polyclonal antibodies, multispecific antibodies (e.g., bispecific antibodies), antibody fragments that can bind an antigen (e.g., Fab′, F′(ab)2, Fv, single chain antibodies, diabodies), and recombinant peptides comprising the forgoing as long as they exhibit the desired biological activity. An “immunoglobulin” or “tetrameric antibody” is a tetrameric glycoprotein that consists of two heavy chains and two light chains, each comprising a variable region and a constant region. Antigen-binding portions may be produced by recombinant DNA techniques or by enzymatic or chemical cleavage of intact antibodies. Antibody fragments or antigen-binding portions include, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, a camelized antibody, a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Monoclonal antibody” refers to an antibody obtained from a population of substantially homogeneous antibodies, i.e., the individual antibodies comprising the population are identical except for possible naturally occurring mutations that may be present in minor amounts.

“Antibody variant” as used herein refers to an antibody polypeptide sequence that contains at least one amino acid substitution, deletion, or insertion in the variable region of the natural antibody variable region domains. Variants may be substantially homologous or substantially identical to the unmodified antibody.

A “chimeric antibody,” as used herein, refers to an antibody containing sequence derived from two different antibodies (see, e.g., U.S. Pat. No. 4,816,567) which typically originate from different species. Most typically, chimeric antibodies comprise human and rodent antibody fragments, generally human constant and mouse variable regions.

A “neutralizing antibody” is an antibody molecule which is able to eliminate or significantly reduce a biological function of an antigen to which it binds. Accordingly, a “neutralizing” antibody is capable of eliminating or significantly reducing a biological function, such as enzyme activity, ligand binding, or intracellular signaling.

“Heavy chain variable region” as used herein refers to the region of the antibody molecule comprising at least one complementarity determining region (CDR) of said antibody heavy chain variable domain. The heavy chain variable region may contain one, two, or three CDR(s) of said antibody heavy chain.

“Light chain variable region” as used herein refers to the region of an antibody molecule, comprising at least one complementarity determining region (CDR) of said antibody light chain variable domain. The light chain variable region may contain one, two, or three CDR(s) of said antibody light chain, which may be either a kappa or lambda light chain depending on the antibody.

As used herein, an antibody that “specifically binds” is “antigen specific”, is “specific for” antigen target or is “immunoreactive” with an antigen refers to an antibody or polypeptide binding agent used herein that binds an antigen with greater affinity than other antigens of similar sequence. In one aspect, the antibody, or fragments, variants, or derivatives thereof, will bind with a greater affinity to human antigen as compared to its binding affinity to similar antigens of other, i.e., non-human, species, but polypeptide binding agents that recognize and bind orthologs of the target are within the scope of the methods.

For example, a polypeptide binding agent that is an antibody or fragment thereof “specific for” its cognate antigen indicates that the variable regions of the antibodies recognize and bind the desired antigen with a detectable preference (e.g., where the desired antigen is a polypeptide, the variable regions of the antibodies are able to distinguish the antigen polypeptide from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of a polypeptide binding agent, e.g. antibody, for use in the methods herein are well known and routinely practiced in the art. For a comprehensive discussion of such assays, see Harlow et al. (Eds), Antibodies A Laboratory Manual; Cold Spring Harbor Laboratory; Cold Spring Harbor, NY (1988), Chapter 6. Antibodies for use in the methods can be produced using any method known in the art.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous.

The term “sample” or “biological sample” refers to a specimen obtained from a subject for use in the present methods, and includes urine, whole blood, plasma, serum, saliva, sputum, tissue biopsies, and cerebrospinal fluid.

The term “therapeutically effective amount” is used herein to indicate the amount of target-specific composition that is effective to ameliorate or lessen symptoms or signs of disease associated with abnormal (e.g., abnormally high or abnormally low) signaling of the signaling complex.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method for determining the efficacy of treatment comprising administering to a patient therapeutic agent in an amount and for a time sufficient to induce a sustained improvement over baseline of an indicator that reflects the severity of the particular disorder.

### Anti-INSR Antibodies

Provided herein are compositions comprising anti-INSR antibodies, for example RZ358, in a stable pharmaceutical formulation. Anti-INSR antibodies that negatively modulate the activity of insulin on the insulin receptor are disclosed in U.S. Pat. Nos. 9,944,698, 10,253,101 and 11,261,247. These negative regulator antibodies allosterically bind to the insulin receptor at a site that does not interfere with insulin binding, but that weakens the binding of insulin to the receptor and reduces signaling through the receptor. This mechanism increases and normalizing blood glucose levels in hyperinsulinemic patients.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

In various embodiments, the disclosure provides an antibody or fragment thereof comprising three heavy chain CDRs having the amino acid sequence set out in SEQ ID NOs: 3-5 and three light chain CDRs have the amino acid sequences set out in SEQ ID NOs: 6-8. In various embodiments, the antibody or fragment thereof binds to i) insulin receptor or (ii) a complex comprising insulin and insulin receptor, or both (i) and (ii).

In various embodiments, the antibody comprises a polypeptide having an amino acid sequence at least about 85%, 86%, 87%, 88%, 89%, 90%, 91%, 92%, 93%, 94%, 95%, 96%, 97%, 98%, 99% or more identical to the heavy chain variable region set out in SEQ ID NO: 1 and an amino acid sequence at least about 85%, 86%, 87%, 88%, 89%, 90%, 91%, 92%, 93%, 94%, 95%, 96%, 97%, 98%, 99% or more identical to the light chain variable region set out in SEQ ID NO: 2, the antibody further comprising at least one, two, three, four, five or all of CDRH1, CDRH2, CDRH3, CDRL1, CDRL2 or CDRL3 set out in SEQ ID NOs: 3-8.

In various embodiments, the anti-INSR antibody or fragment thereof comprises a heavy chain variable region amino acid sequence is set out in SEQ ID NO: 1, and a light chain variable region amino acid sequence is set out in SEQ ID NO: 2.

In exemplary instances, the anti-INSR antibody comprises a heavy chain comprising the amino acid sequence of SEQ ID NO: 9, a light chain comprising the amino acid sequence of SEQ ID NO: 10.

Also contemplated is an antibody or antibody variant that binds INSR comprising (A) a heavy chain variable domain selected from the group consisting of: (i) a sequence of amino acids at least 80% (e.g., about 85%, about 90%, about 95%, greater than 95%) identical to SEQ ID NO:1; (ii) a sequence of amino acids encoded by a polynucleotide sequence encoding a polypeptide that is at least 80% (e.g., about 85%, about 90%, about 95%, greater than 95%) identical to SEQ ID NO:1; (iii) a sequence of amino acids encoded by a polynucleotide that hybridizes under moderately stringent conditions to the complement of a polynucleotide encoding a polypeptide consisting of SEQ ID NO:1; and (B) a light chain variable domain selected from the group consisting of: (i) a sequence of amino acids at least 80% (e.g., about 85%, about 90%, about 95%, greater than 95%) identical to SEQ ID NO:2; (ii) a sequence of amino acids encoded by a polynucleotide sequence encoding a polypeptide that is at least 80% (e.g., about 85%, about 90%, about 95%, greater than 95%) identical to SEQ ID NO: 2; (iii) a sequence of amino acids encoded by a polynucleotide that hybridizes under moderately stringent conditions to the complement of a polynucleotide encoding a polypeptide consisting of SEQ ID NO: 2; or (C) a light chain variable domain of (A) and a heavy chain variable domain of (A), wherein the antibody or antibody variant specifically binds to i) insulin receptor or (ii) a complex comprising insulin and insulin receptor, or both (i) and (ii).

In various embodiments, the antibody or fragment thereof is a Fab fragment.

In various embodiments, the antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody. In various embodiments, the anti-INSR antibody is an IgG2 antibody.

Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In various embodiments, the antibody, antibody variant or fragment thereof binds to (i) insulin receptor or (ii) a complex comprising insulin and insulin receptor, or both (i) and (ii), with an equilibrium dissociation constant Kd of at least 10⁻⁵, 10⁻⁶, 10⁻⁷, 10⁻⁸, 10⁻⁹, 10⁻¹⁰, 10⁻¹¹, 10⁻¹² M, 10⁻¹³ M, 10⁻¹⁴ M, or 10⁻¹⁵ M or less that is capable of weakening the binding affinity between insulin and insulin receptor by at least about 1.5-fold, optionally up to 1000-fold. In certain embodiments, the antibody is capable of weakening the binding affinity between said insulin and insulin receptor by about 2-fold to 500-fold. In various embodiments, the antibody increases the EC₅₀ of insulin signaling activity by about 2-fold to 1000-fold, optionally in a pAKT assay.

### Formulations

The present disclosure provides formulations of anti-INSR antibody RZ358 that are stable over long term storage and at stressed conditions that are useful in the methods described herein.

It is contemplated that the antibody formulation is in liquid or lyophilized form. The formulation may also be a liquid reconstituted from lyophilized form.

In various embodiments, the composition of the present disclosure is a liquid. In certain aspects, the composition has a pH which is less than about 6.5. In some aspects, the pH is about 5.0 to about 6.0 or about 5.1 to about 5.8, or about 5.5 to about 5.9, e.g., about 5.0, about 5.1, about 5.2, about 5.3, about 5.4, about 5.5, about 5.6, about 5.7, about 5.8, about 5.9 or about 6.0.

In various embodiments, the formulation comprises one or more of surfactants, stabilizers, amino acids, antioxidants and/or buffering agents, in order to minimize aggregation, oxidation and other protein degradation during storage. Exemplary stabilizers include surfactants and sugar alcohols. Exemplary anti-oxidants include methionine, sugar alcohols, and histidine. Exemplary buffering agents include histidine HCl.

In various embodiments, the composition comprises histidine. In various embodiments, the histidine is at a concentration of from about 4 mM to about 25 mM, for example about 5 mM to 20 mM, about 10 mM to about 20 mM, or about 8 mM to 15 mM, or about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM. In various embodiments, the histidine is at a concentration of about 4±1 mM.

In certain embodiments, the composition comprises histidine HCl. In various embodiments, the histidine HCl is at a concentration of from about 4 mM to about 25 mM, for example about 5 mM to 20 mM, about 10 mM to about 20 mM, or about 8 mM to 15 mM, or about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM. In various embodiments, the histidine HCL is at a concentration of about 6±1 mM.

In various embodiments, the composition comprises methionine. In various embodiments, the methionine is at a concentration of from about 4 to about 25 mM, for example about 5 mM to 20 mM, about 10 mM to about 20 mM, or about 8 mM to 15 mM, or about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM. In various embodiments, the methionine is at a concentration of about 10±2 mM.

The composition herein may further comprise a sugar alcohol. Exemplary sugar alcohols include sorbitol, mannitol, trehalose, and sucrose. In various embodiments the formulation comprises a sugar alcohol which is sorbitol. In various embodiments, sorbitol is at a concentration of from about 100 mM to about 350 mM, about 150 mM to 350 mM, about 100 mM to 300 mM, about 100 mM to 200 mM, or about 200 mM to about 300 mM. Sorbitol may be at a concentration of about 100 mM, about 125 mM, about 150 mM, about 175 mM, about 200 mM, about 225 mM, about 250 mM, about 275 mM, about 300 mM, about 325 mM or about 350 mM, In various embodiments, the sorbitol is at a concentration of about 270±30 mM.

In various embodiments the formulation comprises a sugar alcohol which is mannitol. In various embodiments, mannitol is at a concentration of from about 100 mM to about 350 mM, about 150 mM to about 350 mM, about 100 mM to about 300 mM, about 100 to about 200 mM, or about 200 mM to about 300 mM. Mannitol may be at a concentration of about 100 mM, about 125 mM, about 150 mM, about 175 mM, about 200 mM, about 225 mM, about 250 mM, about 275 mM, about 300 mM, about 325 mM or about 350 mM. In various embodiments, the mannitol is at a concentration of about 270±30 mM.

In some embodiments, the compositions of the present disclosure comprise a surfactant. Surfactants are surface active agents that are amphipathic (having a polar head and hydrophobic tail). Surfactants preferentially accumulate at interfaces, resulting in reduced interfacial tension. Use of a surfactant can also help to mitigate formation of large proteinaceous particles. In some aspects, the surfactant present in the compositions of the present disclosure is an amphipathic and/or nonionic surfactant. Exemplary surfractants include polyoxyethylene sorbitan fatty acid esters (e.g., polysorbate 20, polysorbate 80), alkylaryl polyethers, e.g., oxyethylated alkyl phenol (e.g., Triton™ X-100), and poloxamers (e.g., Pluronics®, e.g., Pluronic® F68), and combinations of any of the foregoing, either within a class of surfactants or among classes of surfactants. Polysorbate 20 and polysorbate 80 (and optionally mixtures thereof) are contemplated herein. The surfactant in exemplary instances is present in the composition at a concentration of less than or about 0.002% (w/v) to about 0.02% (w/v). For instance, the formulation may comprise about 0.005% (w/v) to about 0.015% (w/v) surfactant, e.g., about 0.005% (w/v), about 0.006% (w/v), about 0.007% (w/v), about 0.008% (w/v), about 0.009% (w/v), about 0.010% (w/v), about 0.011% (w/v), about 0.012% (w/v), about 0.013% (w/v), about 0.014% (w/v), about 0.015% (w/v), about 0.016% (w/v), about 0.017% (w/v), about 0.018% (w/v), about 0.019% (w/v), or about 0.020% (w/v). In exemplary aspects, the formulation comprises about 0.010% (w/v), 0.015% (w/v), or 0.02% (w/v) surfactant.

It is contemplated that the formulation herein comprises the anti-INSR antibody at a concentration from about 20 mg/ml to about 200 mg/mL, from about 50 mg/ml to about 150 mg/mL, or from about 80 mg/mL to about 120 mg/mL. The formulation may comprises the anti-INSR antibody (e.g., RZ358) at a concentration of about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, or about 200 mg/ml.

In various embodiments, the composition comprises about 20-100 mg/mL of an anti-INSR antibody, 0.01% (w/w) polysorbate 20, about 250 to about 300 mM sorbitol, about 5 mM to about 15 mM methionine, and about 5 mM to about 15 mM histidine, wherein the composition has a pH of about 5.8. In various embodiments, the composition comprises about 20-100 mg/mL of an anti-INSR antibody, 0.01% (w/w) polysorbate 20, about 270 mM sorbitol, about 10 mM methionine, and about 10 mM histidine, wherein the composition has a pH of about 5.8. In various embodiments, the antibody is RZ358.

If the formulation is in a form intended for administration parenterally, it can be isotonic with blood (about 300 mOsm/kg osmolality). It is contemplated that the osmolality of the composition is in some aspects, in a range of about 200 mOsm/kg to about 500 mOsm/kg, or about 225 mOsm/kg to about 400 mOsm/kg, or about 250 mOsm/kg to about 400 mOsm/kg, about 250 mOsm/kg to about 350 mOsm/kg, or about 275 to about 375 mOsm/kg. In various embodiments, the formulation has an osmolality of about 275 to about 375 mOsm/kg.

In various embodiments the formulation has a low viscosity. In some embodiments, the formulation is characterized by a viscosity of about 2 cP to about 10 cP, at 25° C., wherein the concentration of the anti-INSR antibody is about 100 mg/ml or less.

In various aspects, the composition of the present disclosure is stable and can endure long term storage at refrigerated temperatures. It is contemplated that less than about 5% (e.g., less than about 4%, less than about 3%, less than about 2%, less than about 1%) of the antibody is degraded after about 1 month to about 3 months of storage at about 2° C. to about 8° C., (e.g., about 2° C., about 4° C., about 6° C., about 8° C.). In various embodiments, less than about 10% (e.g., less than about 9%, less than about 8%, less than about 7%, less than about 6%, less than about 5%, less than about 4%, less than about 3%, less than about 2%, less than about 1%) of the antibody is degraded after 36 months, after 48 months, or after 60 months of storage at about 2° C. to about 8° C. as determined by CE-SDS. In various embodiments, more than about 90%, or about 95% of the antibody is intact after 24 months of storage at about 2° C. to about 8° C., as determined by CE-SDS.

In various embodiments, the composition of the present disclosure is stable and can endure short term storage under stressed storage conditions. Optionally, less than about 5% (e.g., less than about 4%, less than about 3%, less than about 2%, less than about 1%) of the antibody is degraded after about 1 month to about 3 months of storage at about 38° C. to about 42° C., (e.g., about 38° C., about 39° C., about 40° C., about 41° C., about 42° C.).

In various embodiments, after long term storage at about 2° C. to about 8° C., at least 90% of the antibody, or at least about 95% of the antibody, is in monomer form, optionally as detected by SE-HPLC. In various embodiments, less than about 3%, 2.5%, 2%, 1.5%, or 1% of the impurities are high molecular weight species, optimally as detected by SE-HPLC. In various embodiments, after long term storage, less than about 15% of the antibodies are oxidized species, optionally as detected by HIC-HPLC.

In various embodiments, after long term storage at about 2° C. to about 8° C., the antibody maintains 80-120% relative potency compared to a reference antibody, optionally as measured by pAKT assay. In various embodiments, the reference antibody has the heavy chain variable region amino acid sequence set out in SEQ ID NO: 1 and the light chain variable region amino acid sequence set out in SEQ ID NO: 2. In various embodiments, the reference antibody has the heavy chain amino acid sequence set out in SEQ ID NO: 9 and the light chain amino acid sequence set out in SEQ ID NO: 10.

In various embodiments, the composition is provided for storage or use, e.g., in a single-use vial, single-use syringe, or glass, glass-lined, or glass-coated primary container. In various embodiments, the composition is contained in glass vials or syringes for storage, e.g., long-term storage, at about 2° C. to about 8° C. or storage at higher temperatures (e.g., about 25° C., about 30° C., about 40° C.).

In various embodiments, the composition is administered subcutaneously to a subject, and is isotonic with the intended site of administration.

### Methods of Making

Methods of making the composition of the present disclosure are further provided herein. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells.

For recombinant production of the antibody or antibody fragment, the nucleic acid encoding it is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more selective marker genes, an enhancer element, a promoter, and a transcription termination sequence.

Suitable host cells for cloning or expressing the DNA in the vectors herein are prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

Eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present disclosure, particularly for transfection of Spodoptera frugiperda cells.

Examples of useful mammalian host cell lines are Chinese hamster ovary cells, including CHOK1 cells (ATCC CCL61), DXB-11, DG-44, and Chinese hamster ovary cells/-DHFR (CHO, Urlaub et al., Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, (Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y Acad. Sci. 383: 44-68 (1982)); MRC 5 cells; FS4 cells; and a human hepatoma line (Hep G2).

Host cells are transformed or transfected with expression or cloning vectors for antibody production and cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful and preferred for the expression of antibodies that bind the desired antigen.

### Methods of Use

Provided herein is use of the anti-INSR antibody formulations, for example, comprising RZ358, in the treatment of hyperinsulinism/hyperinsulinemia disorders that result from aberrant insulin/INSR signaling and low blood glucose levels.

Exemplary hyperinsulinism disorders include hypoglycemia, insulin sensitivity, cancer, insulinoma, Kaposi's sarcoma, insulin overdose, nesidioblastosis (KATP-HI Diffuse Disease, KATP-HI Focal Disease, or “PHHI”), GDH-HI (Hyperinsulinism/Hyperammonaemia Syndrome (HI/HA), leucine-sensitive hypoglycemia, diazoxide-sensitive hypoglycemia, islet cell dysregulation syndrome, idiopathic hypoglycemia of infancy, Persistent Hyperinsulinemic Hypoglycemia of Infancy (PHHI), Congenital Hyperinsulinism, acute hypoglycemia due to renal failure, chronic hypoglycemia due to renal failure, and hypoglycemia due to chronic kidney disease.

Congenital hyperinsulinism (CHI) comprises a group of genetic disorders that are characterized by recurrent episodes of hyperinsulinemic hypoglycemias due to unregulated secretion of insulin by the pancreatic β-cells (Arnoux J., et al. Orphanet Journal of Rare Diseases 6:63 (2011); Yorifuji T., Ann Pediatr Endocrinol Metab 19:57-68 (2014). CHI is the most common cause of hyperinsulinemic hypoglycemia in neonatal, infant and childhood periods and is usually diagnosed within the first two years of life. Histopathologically, CHI can present in either diffuse or focal forms. In the diffuse form, all pancreatic β-cells are affected, whereas in focal forms, lesions of abnormal β-cells are (usually) restricted to small areas of the pancreas. The most common known causes of CHI are loss-of-function mutations in the genes encoding SUR1 and Kir6.2, subunits of the ATP-sensitive potassium channel (KATP channel), involved in the secretion of insulin in pancreatic β-cells.

There are currently only a few treatment approaches for persistent Congenital Hyperinsulinemia (CHI) [Arnoux J., et al. Orphanet Journal of Rare Diseases 6:63 (2011); Yorifuji T., Ann Pediatr Endocrinol Metab. 19:57-68 (2014)]. Diazoxide, a KATP channel activator, inhibits insulin secretion in pancreatic β-cells. The most frequent adverse effect is hypertrichosis (hirsutism). Other side effects include sodium and fluid retention which may precipitate congestive heart failure. Diazoxide is generally ineffective for those patients that have CHI due to KATP channel mutations, one of the most common causes of CHI. Octreotide is a somatostatin analog that inhibits insulin release. Although not approved for CHI, octreotide is utilized for diazoxide-unresponsive CHI. It is administered subcutaneously (SC) as multiple daily injections or continuously with a pump, or intravenously (IV) because of its short half-life (1 to 2 hours). Common adverse events include gastrointestinal symptoms and gall bladder complications. Partial pancreatectomy is an option for focal lesions and may be curative in a majority of the cases. However, lesions are not always visible or palpable at sites indicated by preoperative imaging. Patients with diffuse forms are primarily treated via continuous glucose feeds or off-label medications. Near-total pancreatectomy has been considered as a treatment, but this is characterized by a high risk of diabetes.

Iatrogenic hypoglycemia describes the condition and effects of low blood glucose caused by administration of either excessive insulin or its analogues, or medications that stimulate endogenous insulin secretion. Iatrogenic hypoglycemia, fundamentally but not exclusively the result of treatment with an insulin secretagogue or insulin, is a major limiting factor in the glycemic management of diabetes. Iatrogenic hypoglycemia causes recurrent morbidity in most people with T1 DM and many with advanced T2DM, and is sometimes fatal. Recurrent episodes of hypoglycemia impair the body's defenses against subsequent falling plasma glucose concentrations and thus cause a vicious cycle of recurrent hypoglycemia.

Hypoglycemia results in a variety of symptoms including; lack of coordination, confusion, loss of consciousness, seizures, and even death.

Most episodes of mild hypoglycemia are effectively self-treated by ingestion of glucose tablets or other carbohydrate containing drinks or snacks. More severe symptomatic hypoglycemia also can be treated with oral carbohydrate ingestion. However, when the hypoglycemic patient cannot take oral glucose supplements, because of confusion, unconsciousness or other reasons, parenteral therapy is required. As a non-hospital rescue procedure, injection of the hyperglycemic hormone, glucagon, is sometimes employed, either subcutaneously or intramuscularly by the patient himself or an associate of the patient who has been trained to recognize and treat severe hypoglycemia. In a medical setting, intravenous glucose is the standard parenteral therapy.

### Administration and Dosing

Also contemplated by the disclosure are methods of administering an antibody composition as described herein to treat a hyperinsulinemia disorder described herein.

Methods of the disclosure are performed using any medically-accepted means for introducing a therapeutic directly or indirectly into a mammalian subject, including but not limited to injections, infusions, oral ingestion, intranasal, topical, transdermal, parenteral, inhalation spray, vaginal, or rectal administration. The term parenteral as used herein includes subcutaneous, intravenous, intramuscular, and intracisternal injections, as well as catheter or infusion techniques. Administration by, intradermal, intramammary, intraperitoneal, intrathecal, retrobulbar, intrapulmonary injection and or surgical implantation at a particular site is contemplated as well. Suitable delivery devices may include those developed for the delivery of insulin (see, e.g., Owens et al Diabetic Med. 20(11):886-898, 2003; US20140128803; and Peyser et al., Annals NY Acad Sci, 1311:102-123, 2014).

An antibody composition as described herein may be administered daily, every 2 days, every 3 days, weekly, every 2 weeks, every 3 weeks, twice monthly, monthly, every 2 months, every 3 months or every 6 months. In various embodiments, the composition is administered for a period of at least 1 month, 2 months, 3 months, 4 months, 6 months, 9 months, 1 year or more.

In one embodiment, administration is performed at the site of an affected tissue needing treatment by direct injection into the site or via a sustained delivery or sustained release mechanism, which can deliver the formulation internally. For example, biodegradable microspheres or capsules or other biodegradable polymer configurations capable of sustained delivery of a composition (e.g., a soluble polypeptide, antibody, or small molecule) can be included in the formulations useful in the disclosure implanted at the site.

Therapeutic compositions may also be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, or monthly.

The amounts of antibody composition in a given dosage will vary according to the size of the individual to whom the therapy is being administered as well as the characteristics of the disorder being treated. In exemplary treatments, it may be necessary to administer about 0.1 to about 25 mg/kg per dose or per day, or from about 0.05 mg/kg to about 10 mg/kg, from about 0.3 mg/kg to about 6 mg/kg, or from about 0.1 mg/kg to about 3 mg/kg. Exemplary doses include, 0.1 mg/kg, 0.3 mg/kg, 0.5 mg/kg 1 mg/kg, 0.75 mg/kg, 1.0 mg/kg, 1.5 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, or 25 mg/kg. Other doses include 1 mg/day, 2.5 mg/day, 5 mg/day, 10 mg/day, 20 mg/day, 25 mg/day, 50 mg/day, 75 mg/day, 100 mg/day, 150 mg/day, 200 mg/day, 250 mg/day, 500 mg/day or 1000 mg/day. These concentrations may be administered as a single dosage form or as multiple doses or continuously.

Also contemplated in the present methods is the administration of multiple agents, such as an antibody composition described herein in conjunction with a second agent as described herein. Compositions comprising an antibody described herein may be administered to persons or mammals suffering from, or predisposed to suffer from, a condition or disorder to be treated associated with the target polypeptide.

Concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks.

A second agent may be other therapeutic agents, such as anti-diabetic agents, cytokines, growth factors, other anti-inflammatory agents, anti-coagulant agents, agents that will lower or reduce blood pressure, agents that will reduce cholesterol, triglycerides, LDL, VLDL, or lipoprotein(a) or increase HDL, agents that will increase or decrease levels of cholesterol-regulating proteins, anti-neoplastic drugs or molecules.

Exemplary agents include, but are not limited to, insulin, glucagon, acarbose, octreotide, verapamil, diazoxide and other agents useful to treat hypoglycemia or side effects associated with hypoglycemia. In various embodiments, an antibody described herein is administered with insulin and/or glucagon, optionally in a delivery device, e.g., a smart delivery device (see, e.g., US20140128803) or dual sensor/pump (e.g., a bionic pancreas system).

Any of the foregoing antibodies or fragments thereof described herein may be concurrently administered with one or more second agent that is an anti-diabetic agent known in the art or described herein, as adjunct therapy.

A number of anti-diabetic agents are known in the art, including but not limited to: sulfonylureas (e.g., glimepiride, glisentide, sulfonylurea, AY31637); biguanides (e.g., metformin); alpha-glucosidase inhibitors (e.g., acarbose, miglitol); thiazol-idinediones (e.g., troglitazone, pioglitazone, rosiglitazone, glipizide, balaglitazone, rivoglitazone, netoglitazone, troglitazone, englitazone, AD 5075, T 174, YM 268, R 102380, NC 2100, NIP 223, NIP 221, MK 0767, ciglitazone, adaglitazone, CLX 0921, darglitazone, CP 92768, BM 152054); glucagon-like-peptides (GLP) and GLP analogs or agonists of GLP-1 receptor (e.g., exendin) or stabilizers thereof (e.g., DPP4 inhibitors, such as sitagliptin); insulin or analogues or mimetics thereof (e.g., lispro, aspart, glulisine, detemir insulin, egludec insulin, insulin glargine, LANTUS®); and Sodium-glucose Cotransporter-2 (SGLT2) inhibitors (e.g., canagliflozin, dapagliflozin, and empagliflozin).

It is contemplated that the antibody and a second agent may be given simultaneously, in the same formulation. It is further contemplated that the agents are administered in a separate formulation and administered concurrently, with concurrently referring to agents given within 30 minutes of each other.

In another aspect, the second agent is administered prior to administration of the antibody composition. Prior administration refers to administration of the second agent within the range of one week prior to treatment with the antibody, up to 30 minutes before administration of the antibody. It is further contemplated that the second agent is administered subsequent to administration of the antibody composition. Subsequent administration is meant to describe administration from 30 minutes after antibody treatment up to one week after antibody administration.

It is further contemplated that other adjunct therapies may be administered, where appropriate. For example, the patient may also be administered a diet or food plan designed for a hypoglycemic patient, surgical therapy, or radiation therapy where appropriate.

It will also be apparent that dosing may be modified if traditional therapeutics are administered in combination with antibody formulations described herein.

### Kits

The present disclosure also provides a kit including a composition described herein together with a package insert, package label, instructions, or other labeling directing or disclosing any of the methods or embodiments disclosed herein. In certain embodiments, the present disclosure provides kits for producing a single-dose administration unit. In certain embodiments of this disclosure, kits containing single and multi-chambered prefilled syringes (e.g., liquid syringes) are included.

Additional aspects and details of the disclosure will be apparent from the following examples, which are intended to be illustrative rather than limiting.

### EXAMPLES

### Example 1—Formulation of RZ358 Antibody

RZ358 is a fully human IgG2 monoclonal antibody (mAb) that binds with high affinity to the insulin receptor (INSR). The RZ 358 antibody has a molecular weight of approximately 149 kD and a pI at pH of 7.6. Described herein is development of formulations of RZ358 for in vivo administration and stability studies and selection of a unique combination of parenterally approved GRAS excipients aimed to stabilize RZ 358 in liquid storage at 2°-8° C. for up to 2 years. Initially, selection of pH that minimizes aggregation and optimize conformational stability was undertaken.

### Methods

Buffer exchange: The buffer exchanges were performed with a VIVASPIN® ultrafiltration device (PES membrane, 30k MWCO, Sartorius Stedim). The concentration was determined by UV absorption measurement at 280 nm with an extinction coefficient of 1.47 cm²/mg. The concentration adjusted solutions were filtered with a syringe driven filter unit (Millex-MP, PES, 0.22 μm, Millipore). The filtered solutions were filled into appropriate containers and placed under different stress conditions for stability studies.

Mechanical stress: RZ 358 solutions were filled into 2 cc glass vials at half capacity (1.5 ml per vial) and sealed with rubber stopper. The vials were placed horizontally in a THERMOMIXER® R and shaken at 1,000 RPM/25° C. for up to 1 week or until visual changes were observed. Freeze-thaw: the filled samples were placed into a −70° C. freezer for at least 2 hours for freezing and then on lab bench until completely thawed. The cycles were repeated for 5 times.

Thermal stress: RZ 358 samples were placed at 5° C., 30° C. and 40° C. and pulled at specific time points (normally 2 weeks, 1 month, 2 months, 3 months and 6 months) for analysis.

Differential Scanning Calorimetry (DSC): DSC analysis was conducted with the TA Instrument NanoDSC. The capillary cells were washed thoroughly with water and sample buffer. Samples/buffers were degassed with the MicroCal ThermoVac Sample Degassing and Thermostat unit for at least 5 minutes at 18° C. before being loaded into the capillary cells. The samples/buffers were scanned from 15° C. to 95° C. at a scan rate of 1° C./min.

Sub-visible Particulate Matter Analysis: The analysis was performed with the Z2 Coulter Particle Count and Size Analyzer. The 100μ aperture probe was calibrated with the “CC L10 Size Standard” and the count accuracy was checked with the Coulter supplied “Concentration Control”. Sample solution was gently swirled and shaken to make sure the solution was homogenous before transferring 0.8 ml of the sample solution into a clean “3 cc” glass vial (capacity ˜4.1 mL) that contained 3.2 mL 0.2M NaCl solution. Each sample was measured at least 2 times. The lower and upper thresholds were set at 10μ and 25μ respectively.

Dynamic light scattering (DLS): The DLS analysis was conducted with the DYNAPRO® Plate Reader (Wyatt Technologies, Santa Barbara, CA). The samples (30 μL each) were loaded into 384-well non-treated assay plate (Corning, Cat #3540, polystyrene, black with clear flat bottom). The instrument temperature was set at 25° C.

All HPLC analyses were conducted with an Agilent 1100 or 1200 system. The data acquisition and analysis were conducted with the ChemStation software (Rev. B.03.02). The detection was by UV at wavelengths 280 nm and 214 nm.

WCX-HPLC: The weak cation exchange HPLC was performed with the Dionex PROPAC™ WCX10 column (4×250 mm). The injected sample was eluded with a pH gradient. The buffer A was 15 mM NaH2PO4 and buffer B 15 mM Na₂HPO₄. The percentage of buffer B was increased from 25 at the start to 95 at 30 minutes. The column was washed at 95% B for 5 minutes and then decreased to 25% B and equilibrated for 15 minutes before making a new injection. The flow rate was kept at 1 ml/min. The eluting peaks were monitored at UV wavelengths of 280 nm and 214 nm. The 280 nm signal is used for integration and quantification calculations.

SEC-HPLC: The SEC-HPLC was performed with a Tosoh TSK gel SWxl G3000 7.8×300 mm column The injected sample was eluted isocratically with elution buffer: 20 mM sodium phosphate, 0.2M ammonium sulfate, pH 6.8 at a flow rate of 0.7 ml/min. The run time was 25 minutes. Peaks were detected at 280 nm.

HIC-HPLC: The hydrophobic interaction chromatography is performed on a Dionex PROPAC™ HIC-10 (5 μm, 4.6×100 mm) column. The buffer A is 0.5M ammonium sulphate, 50 mM sodium phosphate at pH 7 and buffer B 10% (v/v) acetonitrile in 50 mM sodium phosphate at pH 7. The flow rate is 1 mL/min and the run time 30 (45) minutes with the following gradient:

| Time, min | 0 | 2 | 25 | 33 | 33.1 | 45 |
|---|---|---|---|---|---|---|
| % B | 0 | 0 | 100 | 100 | 0 | 0 |

To understand the effect of formulation pH on the formation of aggregates, the RZ 358 antibody was formulated at 1 mg/mL into 10 mM sodium citrate, 150 mM sodium chloride at pH 4.0, 5.0, 5.5, 6.0, 6.3 and 7.0.

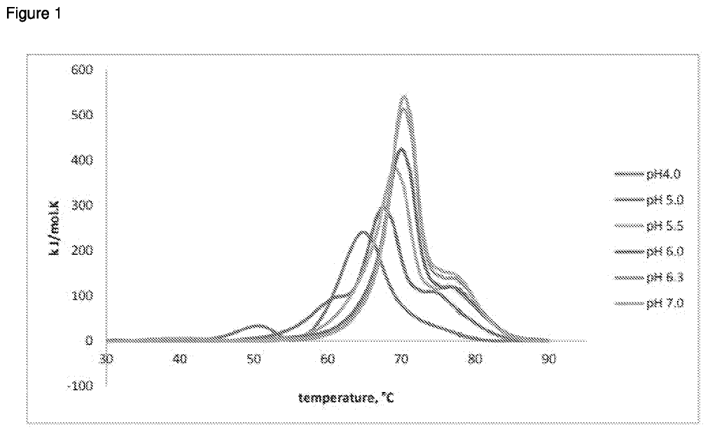

The conformation stability was assessed with DSC. At acidic pH 4, RZ 358 in the above excipients showed a lower temperature transition at 50.7° C. due to the unfolding of the more flexible CH2 domain and a major thermal transition at 64.8° C. of the Fab (FIG. 1). As the formulation pH increased, the CH2 transition temperature increased rapidly and merged into the main Fab transition peak at pH 5.5 and above. The Fab unfolding temperature reaches maximal value once the formulation pH is above 6. The DSC data indicate that the RZ 358 is conformationally more stable when the solution pH is around pH 6 or above.

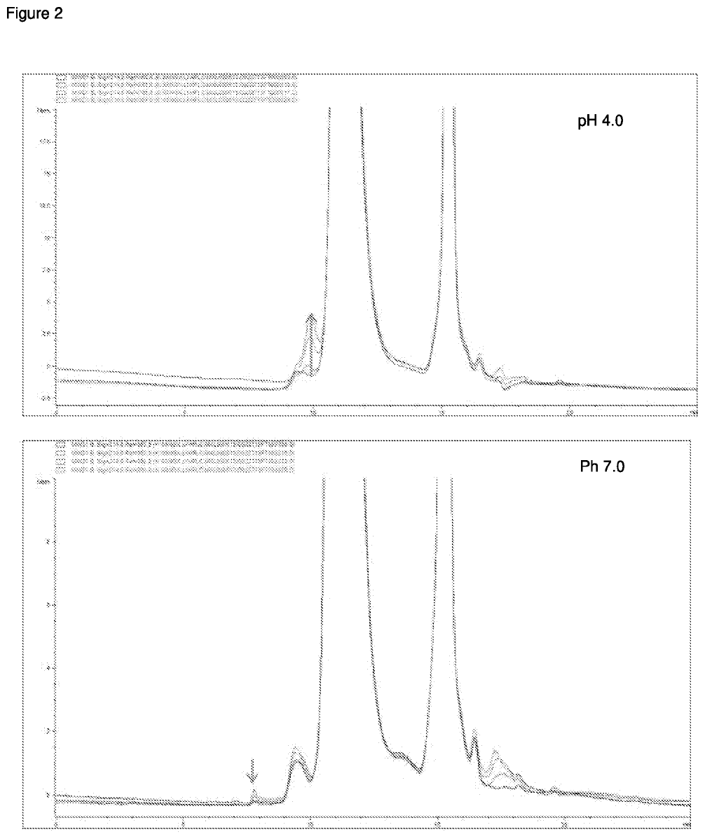

RZ 358 was subjected to five cycles of freeze-thaw and to 40° C. storage conditions. Upon freeze thaw, RZ 358 formed soluble dimers (FIG. 2, upper panel) at acidic pH conditions (pH 4.0 and 5.0) and high molecular weight (HMW) species at neutral pH condition (FIG. 2, lower panel). More optimal stability upon freeze thaw was exhibited in the pH range 5.5 to 6.3.

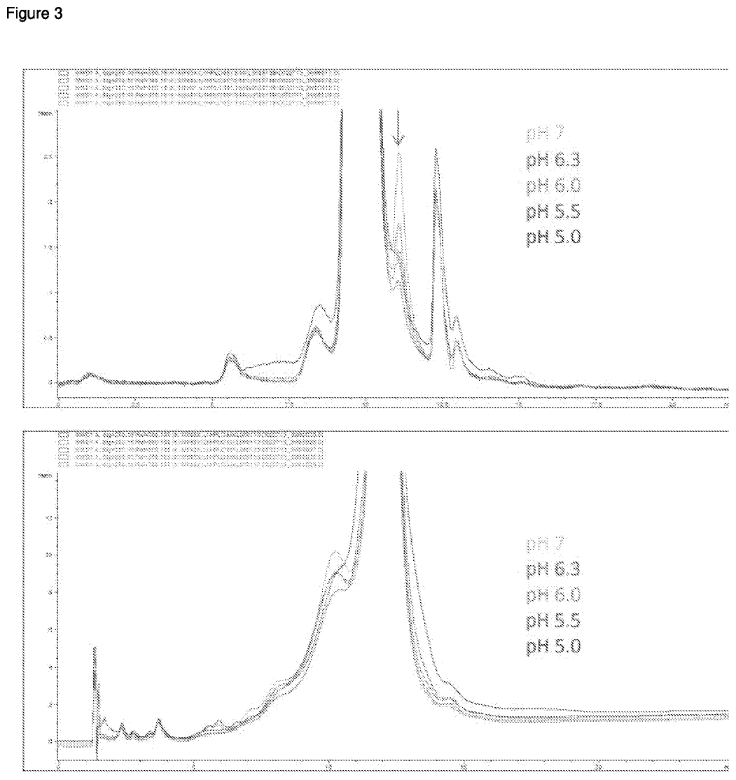

RZ 358 was also incubated at 40° C. for up to 3 months. At pH 4.0, RZ 358 formed soluble aggregates as demonstrated by loss of signal by SEC-HPLC at 2 weeks and by appearance of gel like precipitates after 2 weeks of storage. As the solution pH increased to 5.0 and above, the formation of fragmentation degradants became predominant (FIG. 3, upper panel). The aggregate levels for the stressed 40° C. stability samples (pH 5 to pH 7) at 3 months showed insignificant differences.

RZ 358 fragmentation, as detected by SEC-HPLC, occurred as pH deviated from pH 5.5, both lower (pH 5) and higher pH (7.0). RZ 358 oxidation as measured by degradation pattern from HIC-HPLC (FIG. 3, lower panel) showed a similar trend, with the pH 5.5 formulation showing the least amount of pre-main peaks.

The pH study indicated that the optimal pH for controlling RZ 358 aggregation/fragmentation/oxidation was at approximately pH 5.5.

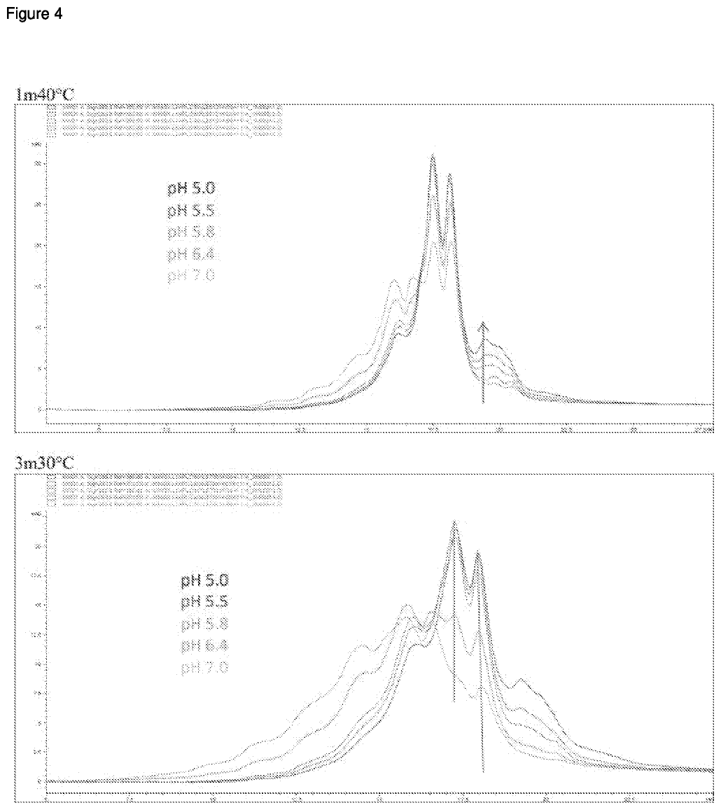

Additional pH studies were carried out based on charge variant distribution. 5 mg/mL RZ 358 was formulated in 10 mM L-histidine/HCl, 10 mM methionine, 270 mM sorbitol with 10 mM L-histidine (free base), 10 mM methionine, 270 mM sorbitol at pH 5.0, 5.5, 5.8, 6.4 and 7.0. Formulation buffer exchange was achieved using VivaSpin ultracentrifugation device. Samples were incubated at 40° C. for one month and at 30° C. for 3 months and analyzed via WCX-HPLC for distribution of the charge variants. Higher pH samples showed faster growth of acidic species. Basic species increased under slightly acidic conditions (FIG. 4).

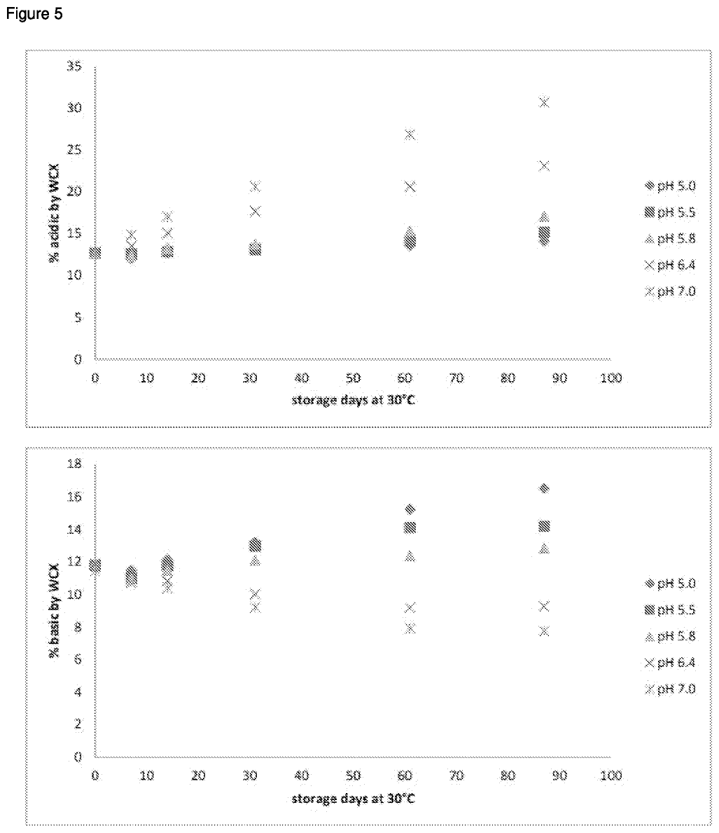

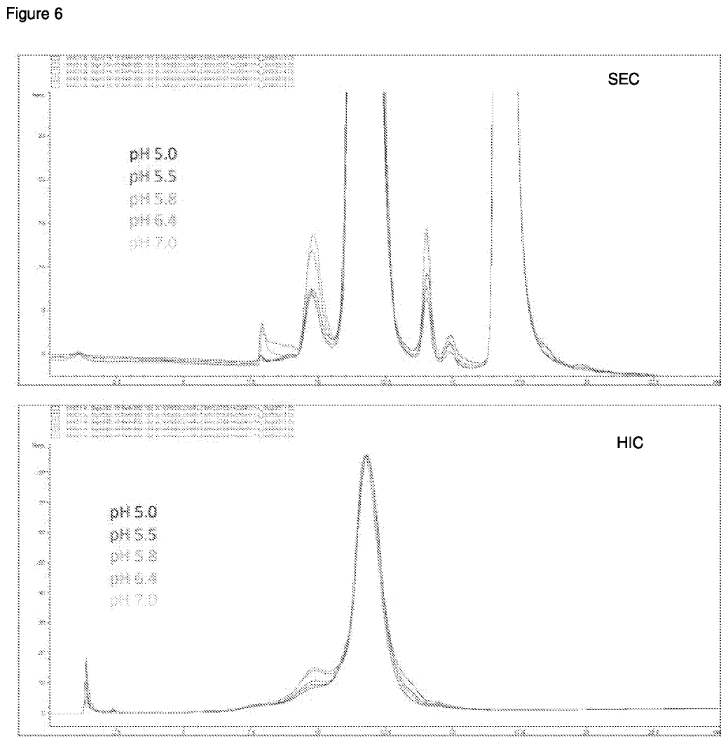

Slightly acidic pH was better for controlling the acidic species and the optimal pH for controlling the basic species formation was around pH 5.8 (FIG. 5). The pH 5.8 formulation was selected to minimize fragment formation (FIG. 6, upper panel), oxidation (FIG. 6, lower panel) and aggregation (FIG. 6, upper panel). It is noteworthy that lower pH formulations generated more species shown as a back shoulder of the HIC main peak.

### Example 2—Analysis of Buffers for RZ358 Formulations

Surfactants can enhance physical stability during freeze thaw and agitation. Experiments were carried out to determine a surfactant that would provide sufficient stability to RZ358 during freeze thaw. RZ 358 was formulated at 2.2 mg/mL into 10 mM L-histidine, 270 mM sorbitol, pH 5.9 with 0, 0.002%, 0.005%, 0.01% and 0.03% polysorbate 20. Each formulated solution of RZ 358 was filled at 1.5 mL into 2 cc Schott glass vials and sealed with West 4432/50 rubber stopper. The filling volume of 1.5 mL is half the maximum capacity of the 2 cc glass vial and was chosen to maximize the air/liquid surface.

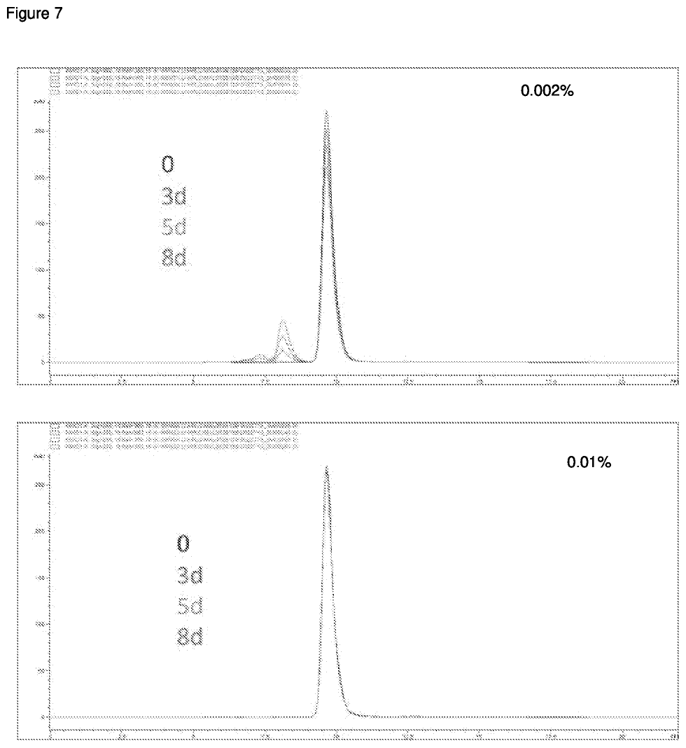

RZ 358 filled vials were exposed to agitation stress by shaking at 1000 RPM up to 8 days at room temperature. Solutions became cloudy upon shaking for less than 3 days in absence of PS20 while it maintained clear for 8 days in the presence of 0.002% or higher PS20. SE-HPLC detected less than 2% of RZ 358 after 3 days of shaking in the absence of PS20. Soluble aggregates did increase with shaking time if the PS20 concentration was 0.002% or 0.005% (FIG. 7). The soluble aggregates showed the least growth at 0.01% PS20 or higher (FIG. 7).

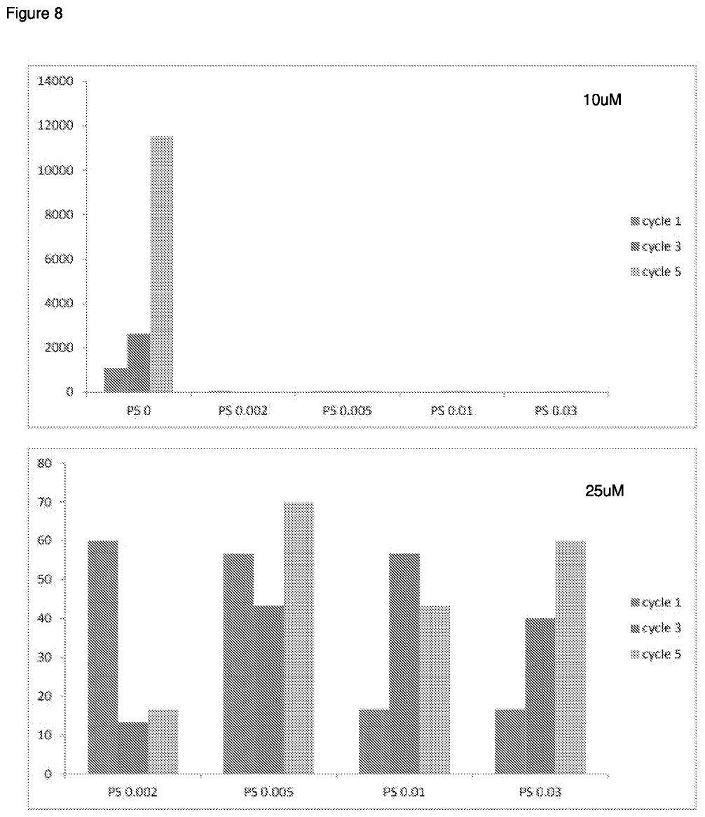

In the absence of the surfactant, freeze-thaw generated numerous visible particles under 25 μm in size, and very few with diameters more than 25 μm. Subvisible particles counts ≤10 μm were significantly reduced at PS20 concentration of 0.002% and greater. No significant impact of PS20 were noted for the particles of ≤25 μm (FIG. 8). The minimum effective PS20 concentration for the prevention of freeze-thaw induced particle formation appeared to be 0.002%. To mitigate agitation and freeze thaw induced aggregation, PS20 concentration of 0.01% was selected for the formulation.

Oxidation of amino acid side chains during purification and storage of protein drug products can lead to instability and breakdown of the therapeutic over time. RZ 358 oxidation control employed uses of scavenger (sugar alcohol) and free radical sink (methionine) in the formulation. Mannitol and sorbitol were tested in the presence of methionine for their effectiveness to minimize RZ 358 oxidation as tested by HIC.

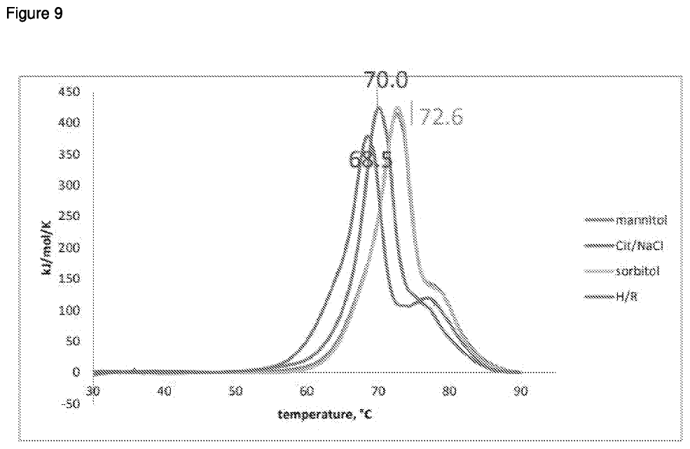

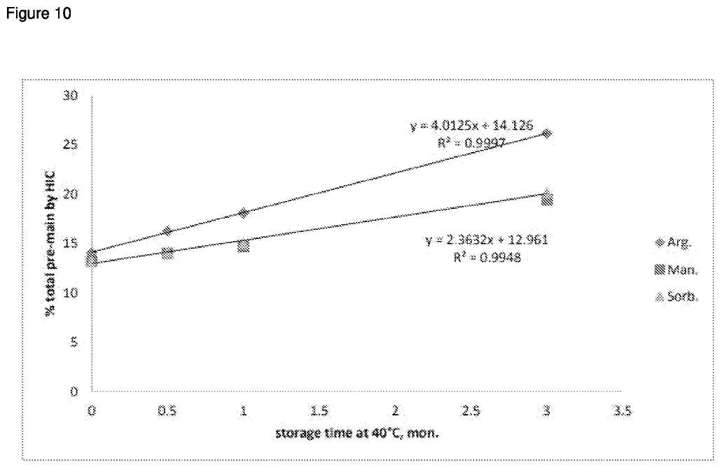

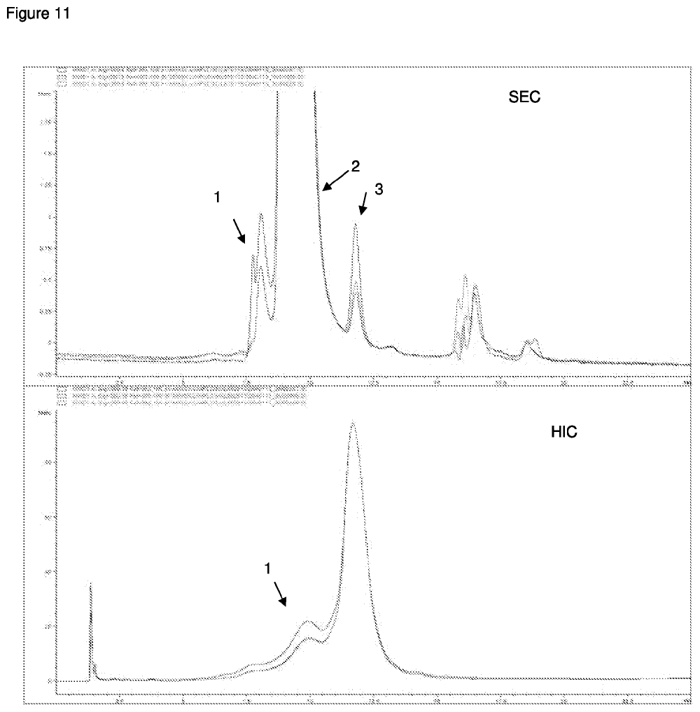

RZ 358 showed better conformational stability in the sugar alcohol/methionine formulation, as showed by higher thermal unfolding temperature in sugar alcohol/methionine formulations than in the arginine formulation (FIG. 9). There appeared no significant difference between the mannitol and sorbitol formulation in terms of thermal unfolding. Oxidation rate was reduced significantly in sugar alcohol/methionine formulations compared with the arginine formulation (FIG. 10 and FIG. 11, lower panel). The two sugar alcohols showed almost equal effectiveness for controlling oxidation. Consistent with the better conformational stability as indicated by the DSC data, the two sugar alcohol formulations showed lower aggregation and lower fragmentation in the stressed temperature storage stability samples, as the SEC-HPLC data demonstrated (FIG. 11, upper panel). Because freeze-thaw cycling led to mannitol precipitation, sorbitol was selected as an excipient for oxidation reduction.

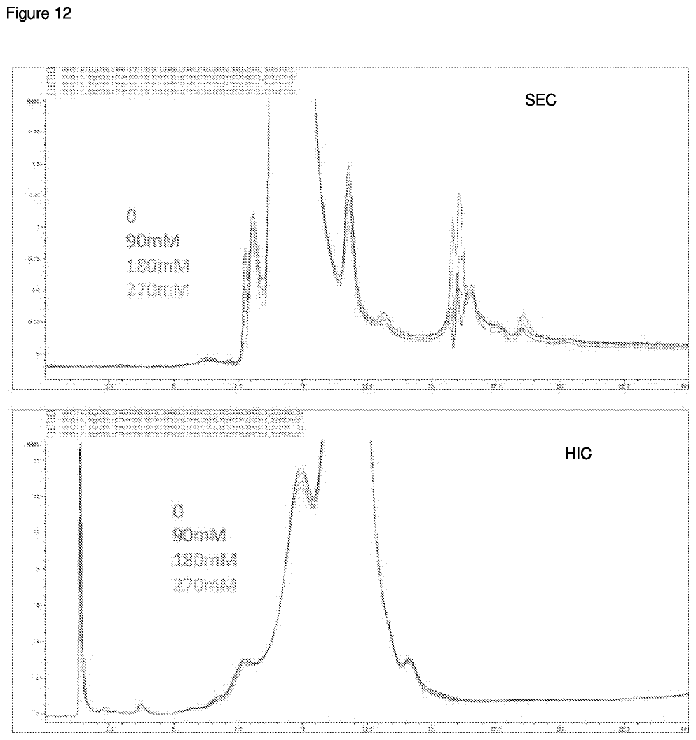

The effect of different ratios of sorbitol to arginine concentration on RZ 358 stability was assessed in 10 mM L-histidine and 10 mM L-methionine at pH 6. RZ 358 was formulated at 5 mg/mL into four different sorbitol/arginine formulations in 10 mM Histidine/methionine at pH 6: [1] 150 mM arginine, [2]90 mM sorbitol/100 mM arginine, [3]180 mM sorbitol/50 mM arginine, and [4] 270 mM sorbitol. The stability data from samples incubated at 40° C. for 2 months indicated that higher sorbitol concentration was more effective in controlling oxidation, aggregation, and fragmentation (FIG. 12).

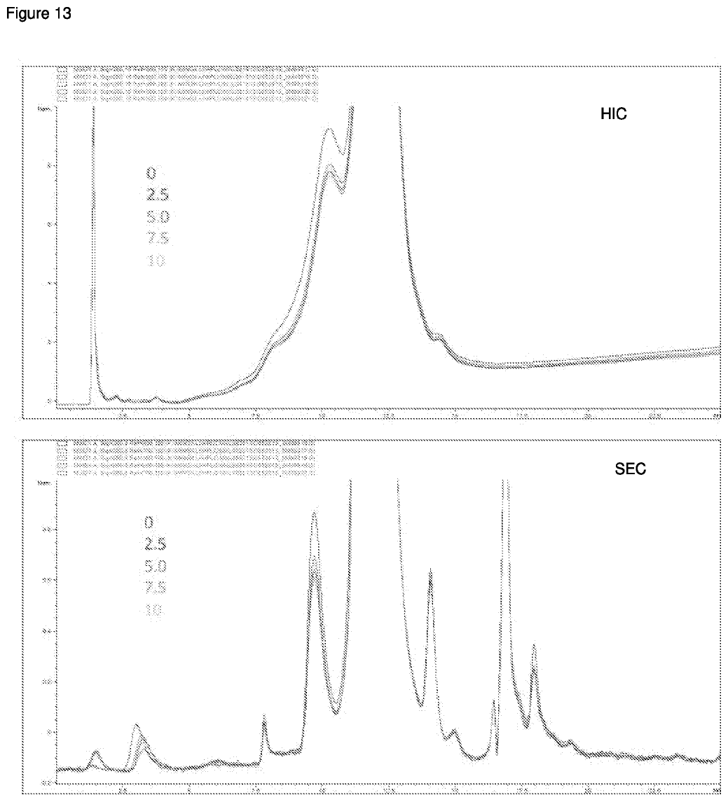

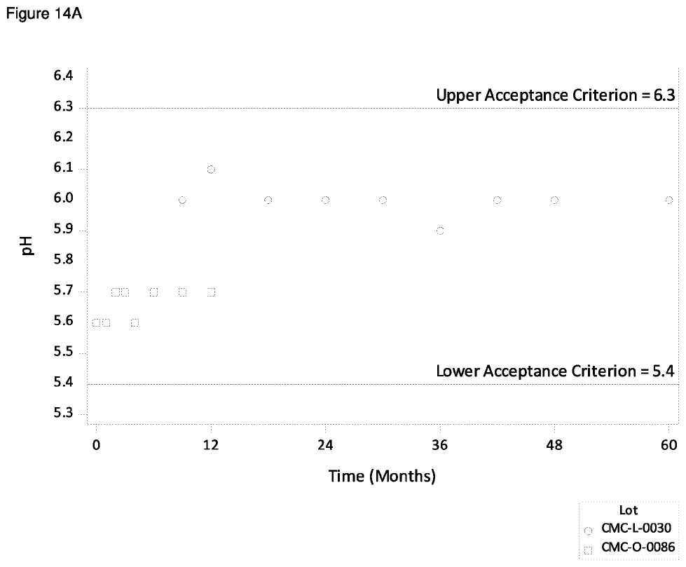

The effect of different methionine concentrations on the stability of RZ 358 was also studied at concentrations of 5 mg/mL RZ 358 in 10 mM L-histidine, 270 mM sorbitol at pH 6 with 0, 2.5 mM, 5 mM, 7.5 mM and 10 mM L-methionine. Formulated samples were stored at 40° C. for up to 3 months. The accelerated stability data indicated that higher methionine concentration led to lower RZ 358 oxidation (FIG. 13, upper panel) and aggregation (FIG. 13, lower panel).

### Example 3—Formulation of RZ358 with Higher Antibody Concentrations

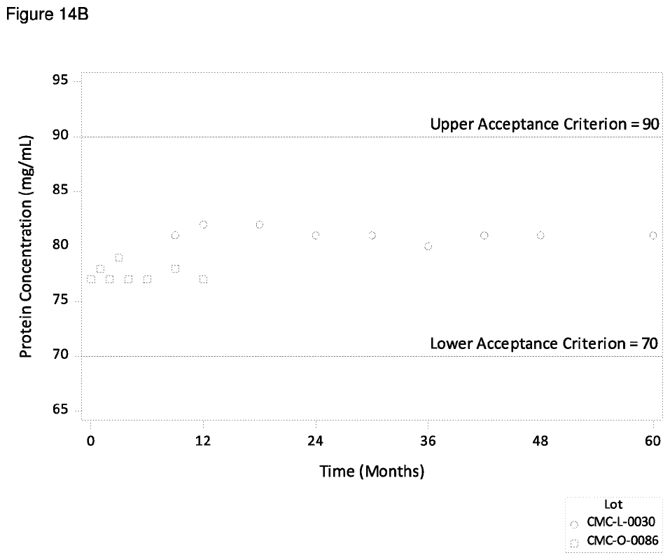

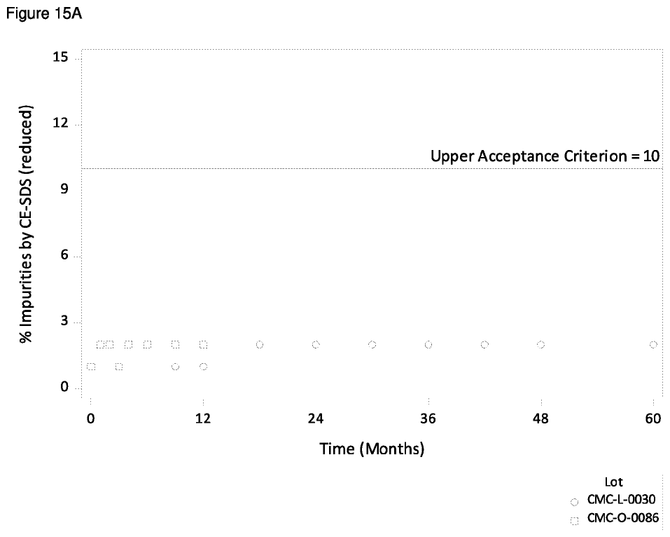

Formulations of RZ358 at higher concentrations were tested, and the long-term stability and potency were assessed. Long term stability at 2° C. to 8° C. of 80 mg/mL RZ358 in 10 mM L-Histidine, 10 mM L-Methionine, 270 mM Sorbitol, 0.01% (w/w) Polysorbate 20, pH 5.8 was measured. Analysis showed that 2 experimental lots were stable between pH 5.4 and pH 6.3 for up to 60 months (FIG. 14A). Protein concentration was also stable in the same storage conditions between 70-90 mg/kg (FIG. 14B). Osmolality measurements of the same lots over the same time frame showed that osmolality was steady at approximately 320-340 mOsm/kg, and could be stable between 286 and 366 mOsm/kg.

Assessment of the % light chain and % heavy chain of the antibody lots after storage for 60 months at 2°-8° C. was assessed by CE-SDS (reduced). The amount of light chain was within the range of 27% to 36% while the amount of heavy chain was within the accepted criteria of 62% to 71%.

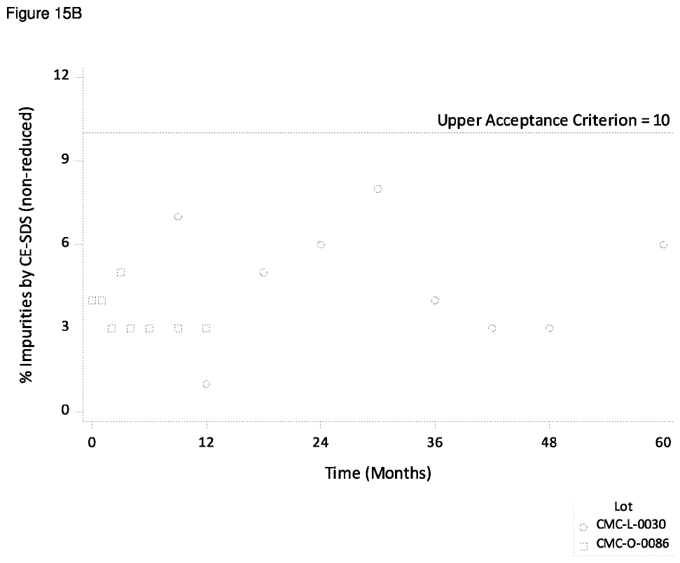

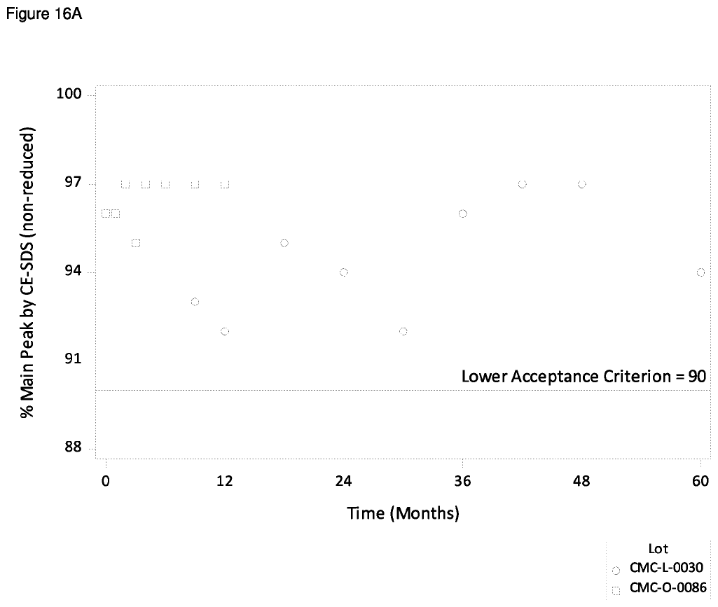

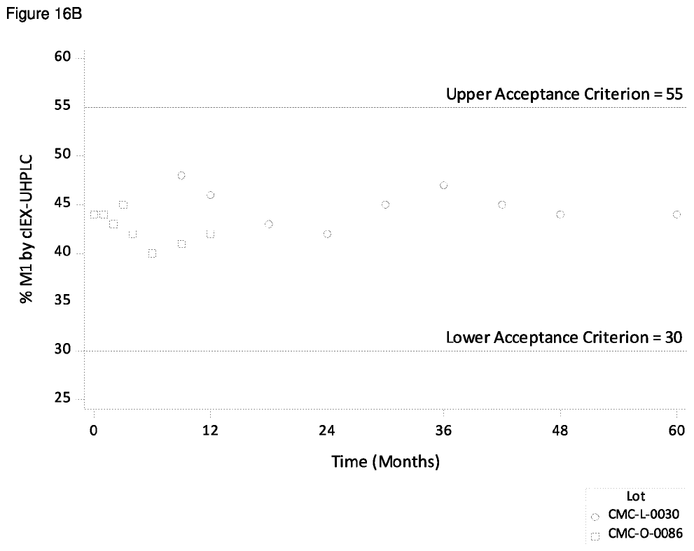

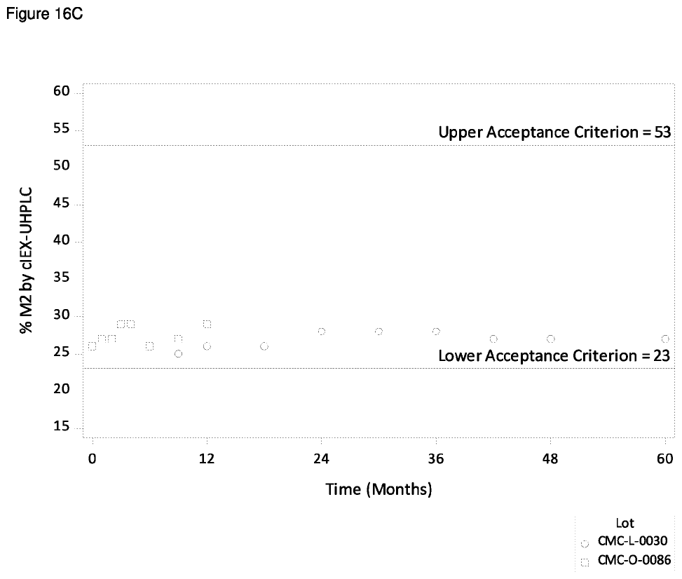

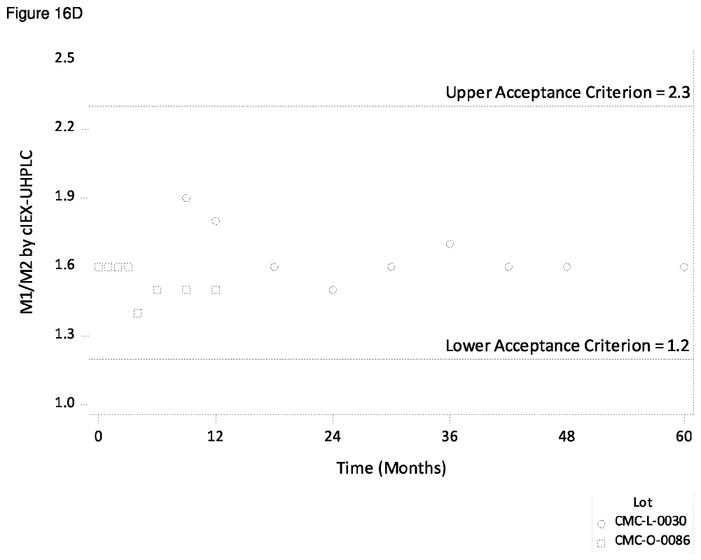

The antibody formulations were measured for impurities and breakdown products after 60 months of storage at 2°-8° C. Total impurities assessed by reduced CE-SDS showed less than 3% impurity in either of the two lots detected over the course of storage time (FIG. 15A). Analysis of impurities by non-reduced CE-SDS showed less than 9% impurities over the storage time (FIG. 15B). Reduced CE-SDS identified the majority of the antibody detectable in the main peak, within the acceptance criteria of 90-100% (FIG. 16A).

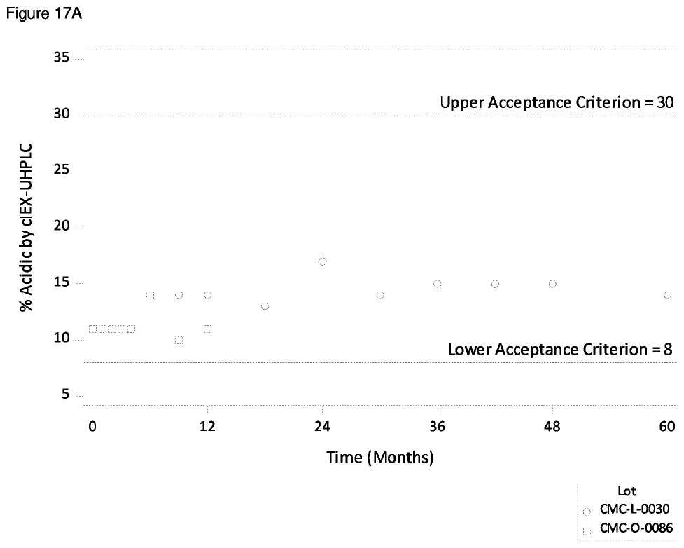

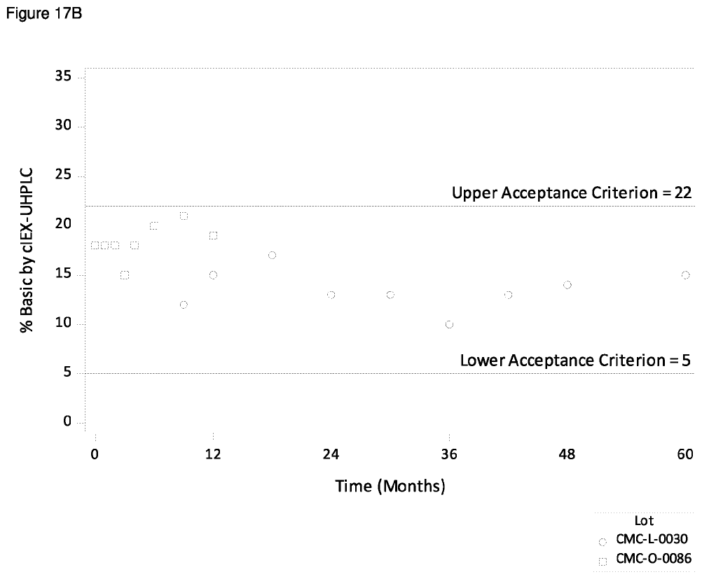

Cation-exchange chromatography analysis showed antibody detected in the main peaks (˜40-50% M1 and ˜30% M2) (FIG. 16B, 16C) with a subset of protein detectable in the acidic peak fraction (less than 20%) (FIG. 17A) and the basic peak fraction (less than 22%) (FIG. 17B).

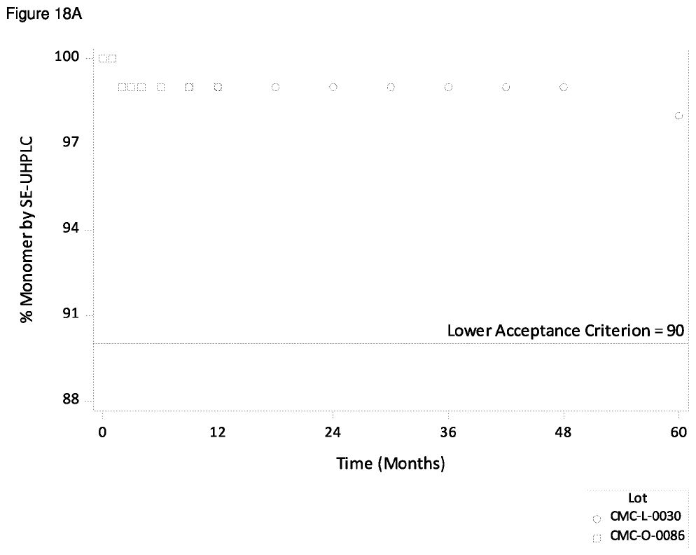

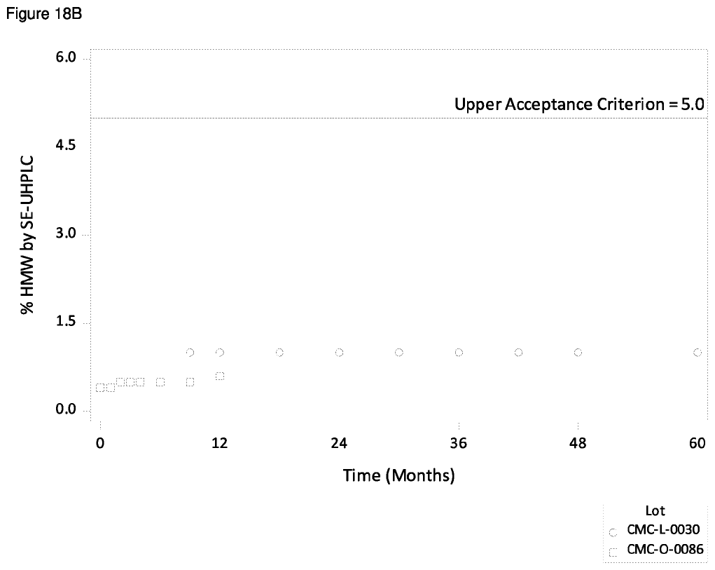

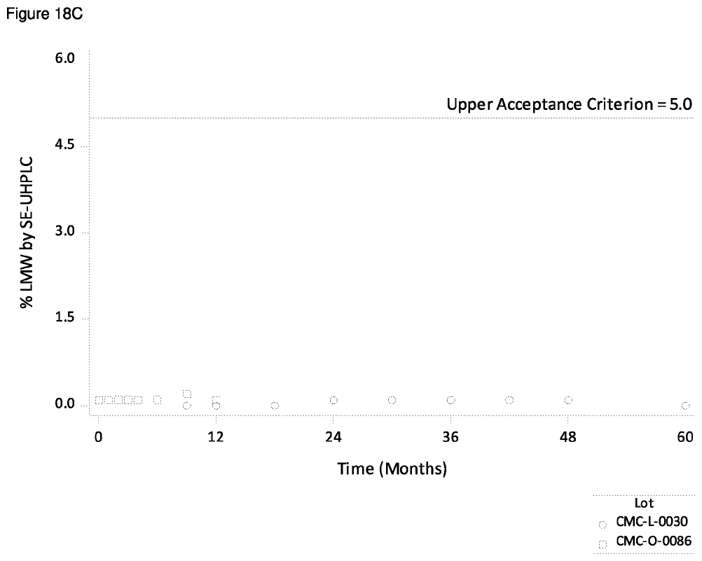

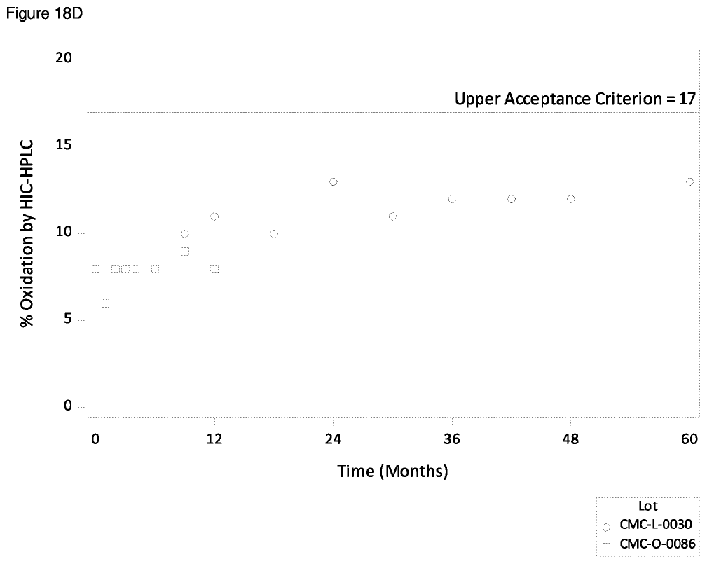

Monomeric, high and low molecular weight, and oxidation species for the 80 mg/ml RZ358 formulation were also determined. SE-HPLC detection of monomeric species show the antibody was over 95% in the monomer form (FIG. 18A), within the 90% acceptance criteria. Additionally, less than 1.5% appeared to be high molecular weight species (FIG. 18B), while little to no low molecular weight species were detected (FIG. 18C). Detection of oxidation species by HIC-HPLC shows that less than 15% of the species were oxidized during storage (FIG. 18D).

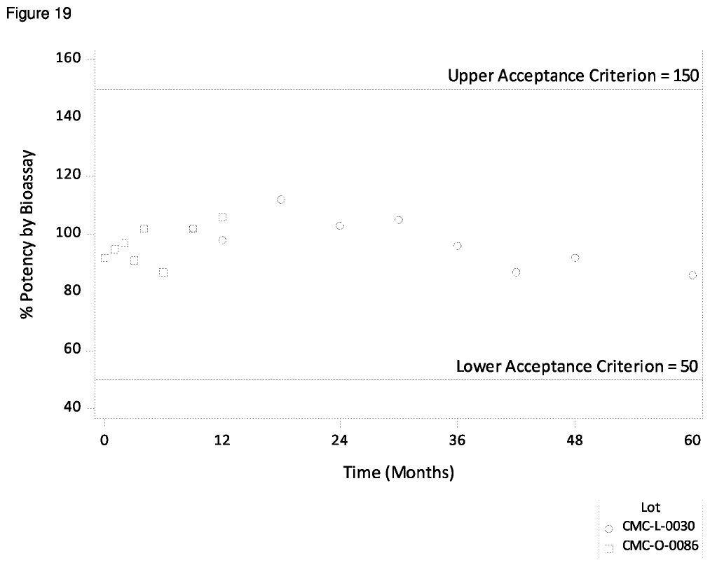

Potency of the stored material was also determined using a cell-based potency assay. Briefly, the bioassay measures the dose dependent inhibition of phosphorylated AKT by RZ 358, by utilizing CHO-K1 hulNS cells that were engineered to express human insulin receptor. To begin, CHO-K1 hulNS cells are insulin starved overnight. Following starvation, the cells are exposed to dose dependent levels of RZ 358 and then stimulated with insulin. The cells are lysed and then phosphorylated Akt and total Akt are quantitated by a sandwich immunoassay that utilizes MSD place that has been pre-coated with capture antibodies for Phospho-AKT (Ser 473) and total Akt. The plate is read and the data of reference standard, test samples and control are fitted to a 4-parameter curve fit model. The final results is a reported as % relative potency against reference standard. FIG. 19 illustrates that the stored RZ 358 maintains good potency over time, with approximately 80-120% relative potency measured over the 60 month storage.

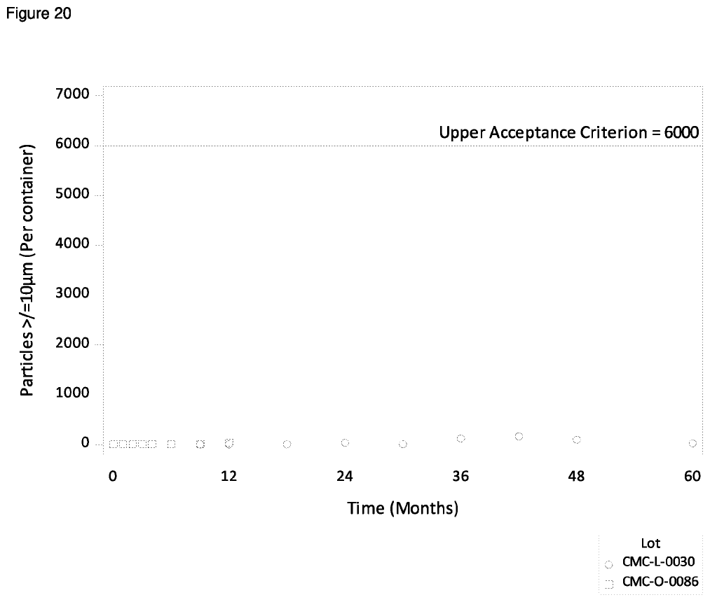

The number of particles formed over time was also measured throughout the storage conditions. Little to no particles were detected in either a 25 μM RZ 358 batch or a 10 μM R358 batch (FIG. 20).

Assessment of surfactant levels on formulation stability: RZ 358 was formulated at 100 mg/mL into 10 mM L-histidine, 10 mM methionine, 270 mM sorbitol, pH 5.8 with 0%, 0.002%, 0.004%, 0.006%, and 0.01% polysorbate 20 (Formulations F1-F5, respectively). Each formulated solution of RZ 358 was sterile filtered and filled at 1.3 mL into 2 cc Schott glass vials and sealed with West 4110/40 rubber stopper.

RZ 358 filled vials were exposed to agitation stress by shaking at room temperature at 300 RPM up to 48 hours followed by 1000 RPM for one hour. In an independent arm of the study, samples were exposed to 5 cycles of freeze-thaw from −20° C. to room temperature. The solutions were analyzed by appearance and for presence of subvisible particles by HIAC. The investigation found that presence of PS20 in RZ358 formulations at concentrations of 0.004% and above mitigated turbidity and visible particle formation induced by agitation and freeze thaw stresses while presence of PS20 levels of 0.002% and above controlled visible particulation in unstressed RZ358 samples at T=0 (Table 1). Under the current experimental conditions, all RZ358 formulations examined, regardless of presence of stress, passed the sub-visible particle USP thresholds, however a marked decrease in particle counts was noted in presence of polysorbate of any level both in T=0 and stressed samples (Table 1).

### Table 1. RZ358 Appearance and Sub-Visible Particle Counts as a Function of Agitation and Freeze Thaw Stresses

| | | F1 | F2 | F3 | F4 | F5 |
|---|---|---|---|---|---|---|
| | | T0 | | | | |
| Appearance | | particles | Clear, free from particles | | | |
| 2 | μm/mL | 2050 | 483 | 295 | 507 | 1777 |
| 5 | μm/mL | 371 | 30 | 39 | 56 | 386 |
| 10 | μm/mL | 72 | 7 | 9 | 22 | 82 |
| 25 | μm/mL | 9 | 2 | 0 | 2 | 9 |
| | | Agitation | | | | |
| Appearance | | Opaque, turbid | particles | Clear, free from particles | | |
| 2 | μm/mL | 16200 | 1129 | 407 | 494 | 1575 |
| 5 | μm/mL | 3554 | 126 | 67 | 72 | 282 |
| 10 | μm/mL | 609 | 22 | 20 | 26 | 43 |
| 25 | μm/mL | 20 | 0 | 4 | 0 | 2 |
| | | Freeze Thaw | | | | |
| Appearance | | Opaque, turbid | particles | Clear, free from particles | | |
| 2 | μm/mL | 6866 | 1049 | 1027 | 984 | 1638 |
| 5 | μm/mL | 1155 | 147 | 93 | 113 | 238 |
| 10 | μm/mL | 186 | 35 | 15 | 33 | 56 |
| 25 | μm/mL | 7 | 2 | 2 | 4 | 9 |

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

We claim:

1. A composition comprising an antibody that specifically binds insulin receptor (INSR), at least one amino acid or a salt thereof, a surfactant, and a sugar alcohol, wherein the anti-INSR antibody comprises
(A) a light chain variable domain comprising: (i) a light chain CDR1 sequence comprising the amino acid sequence set forth in SEQ ID NO: 6; (ii) a light chain CDR2 sequence comprising the amino acid sequence set forth in SEQ ID NO: 7; and (iii) a light chain CDR3 sequence comprising the amino acid sequence set forth in SEQ ID NO: 8; and
(B) a heavy chain variable domain comprising: (i) a heavy chain CDR1 sequence comprising the amino acid sequence set forth in SEQ ID NO: 3; (ii) a heavy chain CDR2 sequence comprising the amino acid sequence set forth in SEQ ID NO: 4, and (iii) a heavy chain CDR3 sequence comprising the amino acid sequence set forth in SEQ ID NO: 5.

2. The composition of claim 1, wherein the at least one amino acid or salt thereof is selected from the group consisting of histidine, histidine HCL, methionine, and arginine.

3. The composition of claim 1 or 2, wherein the amino acid is histidine.

4. The composition of claim 2 or 3, wherein the histidine is at a concentration of from about 4 mM to about 25 mM or about 10 mM to about 20 mM.

5. The composition of any one of claims 2 to 4, wherein the histidine is at a concentration of about 4±1 mM.

6. The composition of claim 1 or 2, wherein the amino acid is histidine HCL.

7. The composition of claim 2 or 6, wherein the histidine HCL is at a concentration of from about 4 mM to about 25 mM or about 10 mM to about 20 mM.

8. The composition of claim 2, 6 or 7, wherein the histidine HCL is at a concentration of about 6±1 mM.

9. The composition of claim 1 or 2, wherein the amino acid is methionine.

10. The composition of claim 2 or 9, wherein the methionine is at a concentration of from about 4 mM to about 25 mM or about 10 mM to about 20 mM.

11. The composition of claim 2, 9 or 10, wherein the methionine is at a concentration of about 10±2 mM.

12. The composition of any one of claims 1 to 11, wherein the surfactant is a polysorbate.

13. The composition of claim 12, wherein the polysorbate is polysorbate 20, polysorbate 80, or a mixture thereof.

14. The composition of any one of claims 1 to 13, comprising the surfactant at a concentration of about 0.002% (w/v) to about 0.02% (w/v).

15. The composition of claim 14, comprising about 0.005% (w/v), 0.010% (w/v), 0.015% (w/v), or 0.02% (w/v) surfactant.

16. The composition of any one of claims 1 to 15, comprising about 0.01% (w/v)±0.0025% (w/v) surfactant, optionally wherein the surfactant is polysorbate 20 or polysorbate 80 or a mixture thereof.

17. The composition of any one of claims 1 to 16, wherein the sugar alcohol is selected from the group consisting of sucrose, sorbitol and mannitol.

18. The composition of any one of claims 1 to 17, wherein the sugar alcohol is sorbitol.

19. The composition of claim 18, wherein the sorbitol is at a concentration of from about 100 mM to about 350 mM or about 200 mM to about 300 mM.

20. The composition of claim 18 or 19, wherein the sorbitol is at a concentration of about 270±30 mM.

21. The composition of any one of claims 1 to 17, wherein the sugar alcohol is mannitol.

22. The composition of claim 21, wherein the mannitol is at a concentration of from about 100 mM to about 350 mM or about 200 mM to about 300 mM.

23. The composition of claim 21 or 22, wherein the mannitol is at a concentration of about 270±30 mM.

24. The composition of any one of claims 1 to 23, wherein the anti-INSR antibody comprises:
(A) a light chain variable domain comprising a sequence of amino acids at least 80% identical to SEQ ID NO: 2; or
(B) a heavy chain variable domain comprising a sequence of amino acids that is at least 80% identical to SEQ ID NO: 1; or
(C) a light chain variable domain of (A) and a heavy chain variable domain of (B).

25. The composition of any one of claims 1 to 24, wherein the anti-INSR antibody comprises a heavy chain comprising the amino acid sequence of SEQ ID NO: 9, and a light chain comprising the amino acid sequence of SEQ ID NO: 10.

26. The composition of any one of claims 1 to 25, wherein the anti-INSR antibody is an IgG2 antibody.

27. The composition of any one of claims 1 to 26, wherein the anti-INSR antibody is present in the composition at a concentration from about 20 mg/ml to about 200 mg/mL.

28. The composition of claim 27, wherein the anti-INSR antibody is present in the composition at a concentration from about 50 mg/ml to about 150 mg/mL.

29. The composition of claim 28, wherein the anti-INSR antibody is present in the composition at a concentration of about 80 mg/mL to about 120 mg/mL.

30. The composition of any one of claims 1 to 29, wherein the composition is a liquid.

31. The composition of any one of claims 1 to 30, wherein the pH is less than about 6.5.

32. The composition of claim 31, wherein the pH is about 5.0 to about 6.

33. The composition of claim 32, wherein the pH is about 5.5 to about 5.9.

34. The composition of claim 33, wherein the pH is about 5.8.

35. The composition of any one of claims 30 to 34, characterized by a viscosity of about 2 cP to about 10 cP, at 25° C., wherein the concentration of the anti-INSR antibody is about 100 mg/ml or less.

36. The composition of any one of claims 30 to 35, wherein the composition is isotonic or has an osmolality in a range of about 200 mOsm/kg to about 500 mOsm/kg, or about 225 mOsm/kg to about 400 mOsm/kg, or about 250 mOsm/kg to about 400 mOsm/kg.

37. The method of any one of the preceding claims, wherein the composition comprises less than about 10% impurities after about 24 months to about 36 months of storage at 2° C. to 8° C. as determined by reduced sodium dodecyl sulfate capillary electrophoresis (rCE-SDS) analysis

38. The composition of any one of the preceding claims, wherein less than 10% of the antibody is degraded after about 24 months to about 36 months of storage at 2° C. to 8° C. as determined by reduced sodium dodecyl sulfate capillary electrophoresis (rCE-SDS) analysis.

39. The composition of any one of the preceding claims, wherein less than 15% of the antibody is oxidized or aggregated after about 3 months of storage at 40° C. as determined by Hydrophobic Interaction Chromatography (HIC) or size exclusion chromatography (SEC).

40. The composition of any one of the preceding claims, wherein the composition comprises less than about 10% degradation products after about 24 months to about 36 months of storage at 2° C. to 8° C. as determined by reduced sodium dodecyl sulfate capillary electrophoresis (rCE-SDS) analysis.

41. The composition of any one of the preceding claims, wherein less than 30% of the antibody is detected in the acidic peak after about 24 months to about 36 months of storage at 2° C. to 8° C. as determined by cIEX-UHPLC analysis.

42. The composition of claim 41, wherein approximately 8-20% of the antibody is detected in the acidic peak after about 24 months to about 36 months of storage at 2° C. to 8° C. as determined by cIEX-UHPLC analysis.

43. The composition of any one of the preceding claims, wherein less than 22% of the antibody is detected in the basic peak after about 24 months to about 36 months of storage at 2° C. to 8° C. as determined by cIEX-UHPLC analysis.

44. The composition of claim 43, wherein about 5-20% of the antibody is detected in the basic peak after about 24 months to about 36 months of storage at 2° C. to 8° C. as determined by cIEX-UHPLC analysis.

45. The composition of any one of the preceding claims, wherein the composition comprises less than 5% high molecular weight species after about 24 months to about 36 months of storage at 2° C. to 8° C. as determined by SE-UHPLC.

46. The composition of any one of the preceding claims, wherein the composition comprises less than 5% low molecular weight species after about 24 months to about 36 months of storage at 2° C. to 8° C. as determined by SE-UHPLC.

47. The composition of any one of the preceding claims, wherein the composition comprises less than 18% of the antibody in oxidized form after about 24 months to about 36 months of storage at 2° C. to 8° C. as determined by HIC-HPLC.

48. The composition of any one of the preceding claims, wherein the potency of the antibody composition is at least about 75% to about 120% after about 24 months to about 36 months of storage at 2° C. to 8° C. as determined by bioassay.

49. A composition comprising about 20-100 mg/mL of an anti-INSR antibody, 0.01% (w/v) polysorbate 20, about 250 mM to about 300 mM sorbitol, about 5 mM to about 15 mM methionine, and about 5 mM to about 15 mM histidine, wherein the composition has a pH of about 5.8.

50. An article of manufacture comprising the composition of any one of the preceding claims optionally, comprising about 0.5 mL to about 5 mL, or about 1 to about 3 mL of the composition.

51. A method for treating a condition associated with hyperinsulinemia or excess insulin signaling in a subject in need thereof comprising administering to the subject a therapeutically effective amount of the composition of any one of claims 1-49.

52. The method of claim 51, wherein the condition is selected from the group consisting of: hypoglycemia, insulin sensitivity, cancer, insulinoma, Kaposi's sarcoma, insulin overdose, nesidioblastosis (KATP-HI Diffuse Disease, KATP-HI Focal Disease, or “PHHI”), GDH-HI (Hyperinsulinism/Hyperammonaemia Syndrome (HI/HA), leucine-sensitive hypoglycemia, diazoxide-sensitive hypoglycemia, islet cell dysregulation syndrome, idiopathic hypoglycemia of infancy, Persistent Hyperinsulinemic Hypoglycemia of Infancy (PHHI), Congenital Hyperinsulinism, acute hypoglycemia due to renal failure, chronic hypoglycemia due to renal failure, and hypoglycemia due to chronic kidney disease.

53. The method of claim 51 or 52 wherein the disease is Congenital Hyperinsulinism.

54. The method of any one of claims 51 to 53, wherein the composition is administered daily, every 2 days, every 3 days, weekly, every 2 weeks, every 3 weeks, twice monthly, monthly, every 2 months, every 3 months or every 6 months.

55. The method of any one of claims 51 to 54, wherein the composition is administered intravenously.

56. The method of any one of claims 51 to 54, wherein the composition is administered subcutaneously.

57. A composition of any one of claims 1-49 for treating a condition associated with hyperinsulinemia or excess insulin signaling in a subject in need thereof.

58. The composition of claim 57, wherein the condition is selected from the group consisting of: hypoglycemia, insulin sensitivity, cancer, insulinoma, Kaposi's sarcoma, insulin overdose, nesidioblastosis (KATP-HI Diffuse Disease, KATP-HI Focal Disease, or “PHHI”), GDH-HI (Hyperinsulinism/Hyperammonaemia Syndrome (HI/HA), leucine-sensitive hypoglycemia, diazoxide-sensitive hypoglycemia, islet cell dysregulation syndrome, idiopathic hypoglycemia of infancy, Persistent Hyperinsulinemic Hypoglycemia of Infancy (PHHI), Congenital Hyperinsulinism, acute hypoglycemia due to renal failure, chronic hypoglycemia due to renal failure, and hypoglycemia due to chronic kidney disease.

59. The composition of claim 57 or 58 wherein the disease is Congenital Hyperinsulinism.
